(19) United States
(12) Patent Application Publication
(10) Pub. No.: US 2025/0352815 A1
(43) Pub. Date: Nov. 20, 2025

(54) METHODS, SYSTEMS, APPARATUSES, AND DEVICES FOR SUPPRESSION OF THE CHRONIC INFLAMMATORY RESPONSE THROUGH VAGAL NERVE SIMULATION

(71) Applicant: BioElectronics Corporation Inc., Frederick, MD (US)

(72) Inventor: John Robert Martinez, Thurmont, MD (US)

(21) Appl. No.: 18/667,971

(22) Filed: May 17, 2024

(51) Int. Cl. A61N 2/00; A61N 2/02

(52) U.S. Cl. CPC A61N 2/006; A61N 2/02

(57) ABSTRACT

A method and an apparatus for facilitating stimulating of vagal nerves in a neck region of a user by utilizing pulsed short-wave therapy technology for purposes of treating chronic inflammation throughout a body of the user. The apparatus includes an apparatus body, a signal generating device, a field generating element, and a powering device. The apparatus body is configured to be placed on a part of the body. The signal generating device is placed in the apparatus body and configured for generating a signal with a signal characteristic. The field generating element is placed in the apparatus body, coupled with the signal generating device, and configured for generating a RF magnetic field with a RF magnetic field characteristic which is shown to suppress an inflammatory response. The powering device is placed in the apparatus body, electrically coupled with the signal generating device, and configured for powering the signal generating device.

### FIELD OF THE INVENTION

Generally, the present disclosure relates to the field of medical and laboratory methods and equipment. More specifically, the present disclosure relates to methods, systems, apparatuses, and devices for facilitating stimulating of vagal nerves of a user in order to suppress chronic inflammation.

### BACKGROUND OF THE INVENTION

Since the earliest stages of physiologic based medical research, the dominant point of view has been that the nervous system and the immune system were distinct entities, both anatomically and functionally. Research undertaken over the past 20 years has shown that this assumption is incorrect.

The peripheral nervous system is traditionally divided into two major subsystems, the somatic nervous system which primarily is used to control voluntary activities of the body, and the autonomic nervous system, which controls involuntary activities in the body. The autonomic system is further divided into the sympathetic system, which prepares the body for “fight or flight” responses, and the parasympathetic system, which regulates bodily activities at rest. The vagus nerve (10th cranial nerve) is the main nerve of the parasympathetic division of the autonomic nervous system. It controls heart rate, gastrointestinal motility and secretion, endocrine and exocrine secretion, glucose production, and numerous other visceral functions. The immune system is also commonly divided into two parts, the innate immune system and the adaptive immune system. The innate immune system responds to pathogens, noxious compounds, or trauma through molecular sensors on immune cell surfaces, or in intracellular compartments. Activation of these sensors leads to a signaling cascade resulting in the increased production of pro-inflammatory cytokines which serve to localize and minimize tissue damage by affecting pathogen clearance, vasodilation, neutrophil recruitment, increased vascular permeability, coagulation factors, etc., in an orchestrated manner referred to as inflammation. Following a successful inflammatory response to invasion, the adaptive immune system serves to ensure that any follow-on invasion by the same agent will be resolved in a more focused and rapid manner.

Inflammation is typically a local and temporary event (lasting from a few days to a few weeks), and following the resolution of the event, immune and physiologic systems return to their resting state. Occasionally, disruption of the innate immune system leads to chronic inflammation, which can last from months to years. Chronic inflammation is associated with a number of syndromes commonly referred to as autoimmune disorders or auto-inflammatory disorders, as well as systemic inflammatory response syndrome. The hallmarks of chronic inflammation are the infiltration of inflammatory cells such as macrophages, lymphocytes, and plasma cells, into the tissue, leading to the progression of tissue damage.

Chronic inflammatory diseases lead to a significant reduction in quality of life as well as being the most significant cause of death in the world. 60% of people, worldwide, die due to chronic inflammatory disease. Currently, over 125M Americans are living with a chronic inflammatory condition. Complications of chronic inflammation include Body pain, joint pain, and muscle pain; Chronic fatigue and insomnia; Depression, anxiety, and mood disorders; Gastrointestinal complications like constipation, diarrhea, and acid reflux; Lack of appetite and weight loss; and Frequent infections.

Treating chronic inflammation represents a significant challenge to modern medicine. There are no highly effective laboratory measures to assess individuals for chronic inflammation and there are few effective interventions, so management of the condition is the most common intervention approach. Management of chronic inflammation commonly includes many dietary and lifestyle changes with the goal of removing the inflammatory triggers such as increasing the intake of anti-inflammatory foods, minimizing the use of antibiotics and anti-inflammatory drugs, increasing exercise, increasing sleep, and decreasing stress. The prognosis for those with chronic inflammatory disease is generally poor.

Over the past 20 years, an alternative perspective on influencing the immune system has been developed, providing a potentially new approach to control excessive innate immune responses that result in tissue damage. Specifically, this perspective arose through the discovery of the inflammatory reflex. In brief, this reflex, similar to reflexes in the somatic nervous system, involves afferent and efferent nerve fibers, specifically, the afferent and efferent arms of the vagus nerve. The afferent arm was first reported in 2000 (Goehler, et al) which showed that vagus afferent fibers innervating the viscera are sensitive to pro-inflammatory cytokines, and communicate the presence of inflammation back to the brainstem.

Subsequently, it was demonstrated that vagus nerve stimulation was sufficient to suppress pro-inflammatory cytokine levels in the periphery. This observation, combined with the knowledge of the role of vagus afferents in the inflammatory response, led to the development of the concept of a centrally regulated inflammatory reflex circuit in the body. Research on the specific molecular mechanisms involved in the inflammatory reflex continues (Caravaca, et al., 2022), but the development of this concept immediately led to investigations on utilizing vagus nerve stimulation as a means of treating non-resolving inflammation. Initial technologies developed for Vagal nerve stimulation (VNS) focused on implanting electrodes along the course of the vagus nerve as it passes through the neck. The first chronic implantable stimulator was used to treat drug-resistant epilepsy in 1988 and required wrapping a fine wire electrode around the left cervical vagus nerve (since the right vagus nerve innervates the sinoatrial node of the heart, high intensity stimulation of the nerve on the right side can influence heart rate). These devices typically produce 20-30 Hz electric pulse stimulation for a duration 30-90 seconds at an intensity sufficient to activate the afferent fibers of the vagus nerve. First approved by the FDA in the 1990s, over 100,000 devices have been implanted in patients worldwide. About 40% of patients find some relief from seizures when using this technology (Johnson & Wilson, 2018). Many patients with implanted VNS devices report improvement in mood, and correspondingly, VNS began to be used to treat depression. This indication was approved by the FDA in 2005.

More recently, it has also been proposed that vagus nerve stimulation could be utilized as an anti-inflammatory treatment. The proposed mechanism is that attenuation of the inflammatory response may occur through a long loop reflex initiated in the vagus afferents, through the autonomic brain stem and forebrain, and then back through the vagus efferent to influence the immune system (i.e. the inflammatory reflex). While studies are ongoing, no reports of significant effects on systemic inflammatory responses have been reported to date. Implantable VNS has a number of limitations. While the implantation is typically completed on an outpatient basis, using general anesthesia, the surgery is inherently risky due to the location of the implantation (requires dissection of the vagus nerve from the carotid artery). In addition, hematomas due to surgical trauma, respiratory complications, vocal cord dysfunction, and obstructive sleep apnea are complications limiting the application of VNS. Furthermore, the cost of implanting a VNS device is very high.

In order to prevent the complications of invasive VNS, and to make VNS more accessible as a therapy, transcutaneous VNS (tVNS) has been developed as a non-invasive technique. tVNS still relies on electric current injection to stimulate the vagus nerve. A typical tVNS device is the gammaCore, which is a handheld appliance with two electrodes that are coated with electrode gel for each use. Stimulation intensity can be set, typically up at 24V and 60 mA, with each session lasting for 120 seconds. The stimulation waveform is a 5 kHz sinusoid with a 1 ms pulse width. The recommendation is to undergo 6-12 treatments per day. It is a limited use device that can provide up to 150 total treatments. Similar to VNS therapy, tVNS is used to treat acute conditions such as tinnitus, migraine headaches, depressive episodes, and seizure prevention (Yap, et al., 2020) rather than chronic conditions. While tVNS removes many of the complications associated with invasive VNS, people find the electrode gel to be messy, accurate positioning of the electrodes during field stimulation of the vagus nerve can be difficult, chronic stimulation is not a viable option, reaction of the skin to the electrode can induce inflammation, and the sensation of the stimulus pulses can be irritating (Jeong, et al., 2022). Correspondingly, research has been undertaken to develop pulsed radio frequency (RF) magnetic field stimulation. The objective in using magnetic stimulation is to impose a relatively high magnitude, transient magnetic field so as to produce relatively large induced electric fields in the tissue surrounding the vagus nerve (typically in the range of 10V/m-20V/m). There is a long history of utilizing time changing magnetic fields to stimulate nerve fibers, for example, patents WO2001078829A2, U.S. Pat. No. 6,527,695B1, and US 20130066392.

As an alternative to using high intensity magnetic fields to produce electric fields in the tissue sufficient to directly stimulate nerve activity, U.S. Pat. No. 8,412,328 B2 utilizes extremely low level magnetic fields (at microTesla levels) at RF frequencies (3-30 MHz) to induce tissue responses. This technology is referred to as pulsed shortwave therapy (PSWT). While radio frequency electromagnetic fields have long been used to heat tissue to enhance tissue repair, the very low levels involved in PSWT preclude any simple explanation of the mechanism. No significant heating arises from the use of such devices, and the electric fields induced into tissue are well below what are believed to be the threshold levels needed to activate nerve or muscle tissue. One suggestion is that there may be a direct magnetic field effect influencing the tissue (perhaps a magneto-biological effect—Henbest, et al., 2004). This technology has been utilized in numerous clinical trials and has been FDA-approved for use in reducing local pain following trauma or surgery. Relative to the other extant stimulation technologies, PSWT is quite inexpensive.

In summary, a wide variety of electrical and magnetic field stimulation techniques have been developed for clinical applications of nerve stimulation and many of these have been applied to stimulation of the vagus nerve. However, in all vagal nerve studies, only brief stimulation durations have been implemented, as longer duration, high intensity electrical stimulation of the nerve has been found to have detrimental side effects. Moreover, chronic inflammation is the predominant clinical challenge, that needs to be addressed, and chronic conditions require a safe and effective, sustained, not intermittent, therapy.

Existing techniques for facilitating stimulating vagal nerves of a user are deficient with regard to several aspects. For instance, many current technologies require surgical insertion for vagus nerve stimulation. As a result, a different technology is needed to provide non-surgical options for vagus nerve stimulation. Furthermore, current technologies use current injection for vagal nerves stimulation which causes tissue inflammation. As a result, a different technology is needed which doesn't use current injection for vagal nerve stimulation for treatment of chronic, systemic inflammation.

Therefore, there is a need for improved methods, systems, apparatuses, and devices for facilitating stimulating of vagal nerves of a user experiencing chronic inflammation that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

This disclosure will describe methods and apparatus for facilitating vagal nerve stimulation over extended time periods for the purposes of treating chronic inflammation. That is, the objective of the intervention is to produce a suppression of the inflammatory response mechanism throughout the body.

Disclosed herein is an apparatus for facilitating stimulation of vagal nerves of a user in the neck region permitting long term therapy of chronic inflammation, in accordance with some embodiments. Specifically, the focus is on modulating vagal nerve activity through exposure of several centimeters (1-10 cm) of the vagal nerve as it passes down the neck below the ear. Accordingly, the apparatus may include an apparatus body, a signal generating device, at least one field generating element, and at least one powering device. Further, the apparatus body may be configured to be placed on at least one part of a body of the user. Further, the at least one part of the body may be associated with at least one vagal nerve. Further, the signal generating device may be placed in the apparatus body. Further, the signal generating device may be configured for generating at least one signal with at least one signal characteristic. Further, the at least one field generating element may be placed in the apparatus body. Further, the at least one field generating element may be coupled with the signal generating device. Further, the at least one field generating element may be configured for generating at least one RF magnetic field with at least one RF magnetic field characteristic based on the generating of the at least one signal. Further, the at least one powering device may be placed in the apparatus body. Further, the at least one powering device may be electrically coupled with the signal generating device. Further, the at least one powering device may be configured for powering the signal generating device. Further, the generating of the at least one signal with the at least one signal characteristic may be based on the powering.

Further disclosed herein is an apparatus for facilitating stimulating of vagal nerves of a user for extended time periods in order to treat chronic inflammation, in accordance with some embodiments. Accordingly, the system may include an apparatus body, a signal generating device, at least one field generating element, and at least one powering device. Further, the apparatus body may be configured to be placed on at least one part of a body of the user. Further, the at least one part of the body may be associated with at least one vagal nerve. Further, the apparatus body may include a neck strap portion and a pendant portion attached to the neck strap portion. Further, the at least one part of the body may include a neck of the user. Further, the neck strap portion may be placed around the neck of the user. Further, the signal generating device may be placed in the apparatus body. Further, the signal generating device may be configured for generating at least one signal with at least one signal characteristic. Further, the at least one field generating element may be placed in the apparatus body. Further, the at least one field generating element may be coupled with the signal generating device. Further, the at least one field generating element may be configured for generating at least one RF magnetic field with at least one RF magnetic field characteristic based on the generating of the at least one signal. Further, the at least one field generating element may be comprised in the neck strap portion. Further, the at least one powering device placed in the apparatus body. Further, the at least one powering device may be electrically coupled with the signal generating device. Further, the at least one powering device may be configured for powering the signal generating device. Further, the generating of the at least one signal with the at least one signal characteristic may be based on the powering. Further, the signal generating device and the at least one powering device may be comprised in the pendant portion.

Further, disclosed herein is an apparatus for facilitating stimulating of vagal nerves of a user for extended durations in order to treat chronic inflammation, in accordance with some embodiments. Accordingly, the apparatus may include an apparatus body, a signal generating device, at least one field generating element, and at least one powering device. Further, the apparatus body may be configured to be placed on at least one part of a body of the user. Further, the at least one part of the body may be associated with at least one vagal nerve. Further, the apparatus body may include a neck strap portion and a pendant portion attached to the neck strap portion. Further, the at least one part of the body may include a neck of the user. Further, the neck strap portion may be placed around the neck of the user. Further, the neck strap portion extends between a first end and a second end. Further, the neck strap portion may include a connecting element. Further, the connecting element may be configured for transitioning a loop formed by the neck strap portion between a continuous loop and a discontinuous loop. Further, the connecting element may include a first connecting portion and a second connecting portion. Further, the first connecting portion may be comprised in the first end of the neck strap portion and the second connecting portion may be comprised in the second end of the neck strap portion. Further, the first connecting portion may be disconnectably connectable to the second connecting portion for the transitioning of the neck strap portion between the continuous loop and the discontinuous loop. Further, the signal generating device may be placed in the apparatus body. Further, the signal generating device may be configured for generating at least one signal with at least one signal characteristic. Further, the at least one field generating element may be placed in the apparatus body. Further, the at least one field generating element may be coupled with the signal generating device. Further, the at least one field generating element may be configured for generating at least one RF magnetic field with at least one RF magnetic field characteristic based on the generating of the at least one signal. Further, the at least one field generating element may be comprised in the neck strap portion. Further, the at least one powering device may be placed in the apparatus body. Further, the at least one powering device may be electrically coupled with the signal generating device. Further, the at least one powering device may be configured for powering the signal generating device. Further, the generating of the at least one signal with the at least one signal characteristic may be based on the powering. Further, the signal generating device and the at least one powering device may be comprised in the pendant portion.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a front view of an apparatus 100 for facilitating stimulating of vagal nerves of a user, in accordance with some embodiments.

FIG. 2 is a front view of the apparatus 100 for facilitating the stimulating of the vagal nerves of the user, in accordance with some embodiments.

FIG. 3 is a front view of the apparatus 100 for facilitating the stimulating of the vagal nerves of the user, in accordance with some embodiments.

FIG. 4 is a front view of the apparatus 100 for facilitating the stimulating of the vagal nerves of the user, in accordance with some embodiments.

FIG. 5 is a front view of the apparatus 100 for facilitating the stimulating of the vagal nerves of the user, in accordance with some embodiments.

FIG. 6 is a front view of the apparatus 100 for facilitating the stimulating of the vagal nerves of the user, in accordance with some embodiments.

FIG. 7 is a front view of the apparatus 100 for facilitating the stimulating of the vagal nerves of the user, in accordance with some embodiments.

FIG. 8 is a front view of the apparatus 100 for facilitating the stimulating of the vagal nerves of the user, in accordance with some embodiments.

FIG. 9 is a rear view of the apparatus 100 for facilitating the stimulating of the vagal nerves of the user, in accordance with some embodiments.

FIG. 10 is a rear view of the apparatus 100 for facilitating the stimulating of the vagal nerves of the user, in accordance with some embodiments.

FIG. 11 is a front view of the apparatus 100 for facilitating the stimulating of the vagal nerves of the user, in accordance with some embodiments.

FIG. 12 is a front view of the apparatus 100 for facilitating the stimulating of the vagal nerves of the user, in accordance with some embodiments.

FIG. 13 is a front view of the apparatus 100 for facilitating the stimulating of the vagal nerves of the user, in accordance with some embodiments.

FIG. 14 is a front view of the apparatus 100 for facilitating the stimulating of the vagal nerves of the user, in accordance with some embodiments.

FIG. 15 is a front view of the apparatus 100 for facilitating the stimulating of the vagal nerves of the user, in accordance with some embodiments.

FIG. 16 is a perspective view of the apparatus 100 for facilitating the stimulating of the vagal nerves of the user, in accordance with some embodiments.

FIG. 17 is a front view of the apparatus 100 for facilitating the stimulating of the vagal nerves of the user, in accordance with some embodiments.

FIG. 18 is a front view of the apparatus 100 for facilitating the stimulating of the vagal nerves of the user, in accordance with some embodiments.

FIG. 19 is a partial rear view of the apparatus body 102 for facilitating the stimulating of the vagal nerves of the user, in accordance with some embodiments.

FIG. 20 is a side view of the apparatus 100 worn by the user, in accordance with some embodiments.

FIG. 21 is a side view of the apparatus 100 worn by the user, in accordance with some embodiments.

FIG. 22 is a perspective view of an apparatus 2200 for facilitating stimulating of the vagal nerves of the user, in accordance with some embodiments.

FIG. 23 is a front view of an apparatus 2300 for facilitating stimulating of vagal nerves of a user, in accordance with some embodiments.

FIG. 24 is a front view of a strap 2402 for receiving an apparatus for facilitating stimulating of vagal nerves of a user, in accordance with some embodiments.

FIG. 25 is a perspective view of an apparatus 2500 for facilitating stimulating of vagal nerves of a user, in accordance with some embodiments.

FIG. 26 is a perspective view of the apparatus 2500, in accordance with come embodiments.

FIG. 27 is a front view of an apparatus 2700 for facilitating stimulating of vagal nerves of a user, in accordance with some embodiments.

FIG. 28 is a front view of an apparatus 2800 for facilitating stimulating of vagal nerves of the user, in accordance with some embodiments.

FIG. 29 is an illustration of an online platform consistent with various embodiments of the present disclosure.

FIG. 30 is a block diagram of a computing device for implementing the methods disclosed herein, in accordance with some embodiments.

FIG. 31 is a graph 3100 illustrating a time course of improvement in average discomfort associated behavior (DAB) level for the study for reversing chronic inflammation in a canine model utilizing pulsed short-wave therapy, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that, does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods, systems, apparatuses, and devices for facilitating stimulating of vagal nerves of a user, embodiments of the present disclosure are not limited to use only in this context.

In general, the method disclosed herein may be performed by one or more devices. For example, in some embodiments, the method may be performed by one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, and so on.

Further, one or more steps of the method disclosed herein may be initiated, maintained, controlled and/or terminated based on a control input received from one or more devices operated by one or more users such as, for example, but not limited to, an end user, an administrator, a service provider, a service consumer, an agent, a broker and a representative thereof. Further, the user as defined herein may refer to a human, or an animal in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods and apparatus for facilitating vagal nerve stimulation over extended time periods for the purposes of treating chronic inflammation. That is, the objective of the intervention is to produce a suppression of the inflammatory response mechanism throughout the body.

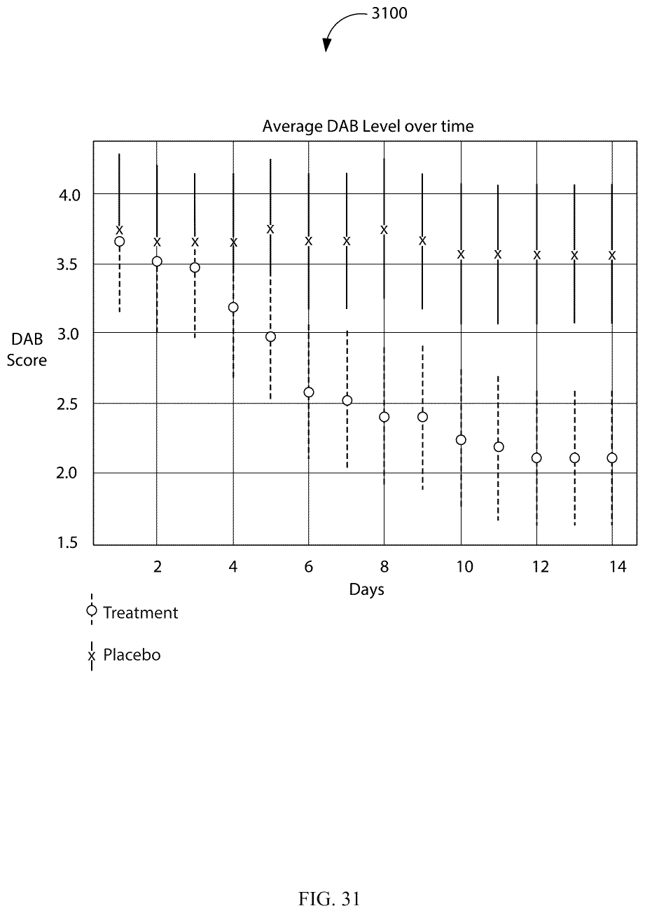

Acting on the reported effectiveness of low level (non-thermal) pulsed RF magnetic fields in limiting pain due to local tissue trauma, a test was conducted to determine whether pulsed shortwave therapy (PSWT) would be effective in reversing chronic inflammation in a canine model of chronic joint inflammation. Specifically, a repurposed FDA-approved pulsed short-wave therapy device was used to expose the vagus nerves in the neck of canines to a low-level RF magnetic field (flux density ˜1-5 microTesla) at a frequency of 27.1 MHz, utilizing 100-microsecond pulses repeated at a rate of 1000 pulses per second. Peak-induced electric fields in the region of the vagus nerve were less than 1V/m, far below the threshold for nerve excitation. Canines, with a veterinarian diagnosed a significant level of chronic discomfort in a forelimb and/or hind limb, were enrolled in a randomized, placebo controlled, double blind study which involved two weeks of continuous (24/7) therapy. 26 animals were randomized into the treatment arm, and 23 animals into the placebo control arm of the study. The assessment involved 8 behavioral factors scored on an 11-point scale. These eight factors represented a whole-body behavioral assessment (here-after referred to as a discomfort-associated behavior (DAB) score) of the inflammatory response in the animal and focused on deviations from normality in breathing, vision, ambulation, activity levels, appetite, attitude, posture, and sensitivity to touch. PSWT exposure was found to be highly efficacious over the course of the study. Following two weeks of exposure, the average DAB score in the treatment group declined from 4.25 to 2.2 while the placebo-treated animals demonstrated no change in their DAB scores over the two-week study duration, as shown in FIG. 31. This difference was statistically significant (p<0.05) within eight days of the start of treatment, and clinically significant improvement (2×) was observed by the end of the study, consistent with significant suppression of the inflammatory responses in the animals. Placebo-treated animals demonstrated no change in their discomfort scores over the two-week study duration. These results demonstrate that a specific pulsed radio-frequency magnetic field exposure (˜1 microTesla, 27 MHz, 100 microsecond pulses at 1 KHz), applied in the region of the vagus nerve, continuously for a period of at least 8 days can produce significant inhibition of systemic inflammatory responses in a mammalian system. These results are consistent with the hypothesis that the RF magnetic field exposure is modulating vagus nerve activity, and correspondingly, the inflammatory reflex, resulting in suppression of the hyper-inflammatory responses in the body. The present disclosure proposes that similar effects will be observed in human trials of this new concept.

The present disclosure describes the use of very low level (<1 microTesla) radio frequency (5-50 MHz) magnetic fields to modulate the activity of the vagus nerve in humans or other animals. Through appropriate orientation of the RF magnetic field, the exposure of the vagus nerve can be optimized so as to maximize the physiologic response. The predominant clinical application of this invention is in the treatment of chronic, inflammatory disease.

The vagus nerves are located near the front of the neck, between the carotid arteries and the jugular veins. They progress along an approximate 10 cm distance in the neck at a depth from the skin surface to the vagus nerves of approximately 2.5 cm, in the average-sized human. Distance from the back of the neck to the vagus nerves is approximately 7 cm in the average-sized human. Canine necks are similar in size to that of the human, while feline necks are significantly smaller. A pulsed shortwave therapy (PSWT) RF field generator is used to produce sufficient power to affect vagus nerve activity. The device is powered by a 3V non-rechargeable battery with an energy capacity of approximately 220 mA-Hr. The generator utilizes an ASIC chip technology to create the stimulation waveform and is approximately 3 cm in diameter and 0.6 cm in thickness. A pulsed shortwave therapy device can be modified to fit around the neck of an animal or human so as to optimally expose the vagus nerves to radiofrequency magnetic field exposure. In one application, a wire loop is designed to match the circumference of the neck, and a connector is inserted into the antenna to permit ease of placement and removal. Alternatively, an earphone concept for exposing the vagus nerve, as it passes in front of the ear, to RF magnetic fields can be used. For effective modulation of the vagus nerves, it is, therefore, necessary to ensure that the antenna is designed appropriately so that the RF magnetic field penetrates to the level of the vagus nerve with an intensity sufficient to modulate vagus nerve activity.

Further, the present disclosure describes a new, and non-obvious, use of pulsed shortwave therapy technology. While the existing technology is utilized to modulate nerve activity in the periphery of the body in order to reduce localized pain, by exposing the vagus nerve where it enters the brainstem, we have shown that a suppression of chronic inflammatory responses can be obtained.

The specific device utilized to modulate vagus nerve activity operates at 27.1 MHz, which is square wave modulated at 1 KHz, utilizing a 100 microsecond pulse width. The signal generator utilized in preliminary work was capable of delivering 0.5 microwatts of power, for over 700 hours. The generator is packaged on a 3 cm diameter circuit board, and the package is approximately 0.6 cm in thickness. The generator has two external connections which power a current loop.

The vagus nerves are located approximately 1 inch (2.5 cm) below the surface of the skin of the human neck, specifically, the region of the neck immediately to the left and right of the trachea. From the back surface of the neck, the vagus nerves are approximately 2.5 inches (6.25 cm) below skin level. For effective modulation of the vagus nerves, it is therefore necessary to ensure that the antenna is designed appropriately so that the RF magnetic field penetrates to the level of the vagus nerve with an intensity sufficient to modulate vagus nerve activity as it passes down the length of the neck.

Measurements of RF magnetic field flux density at the site of the vagus nerves demonstrate that a flux density of at least 0.5 microTesla peak (0.35 microTesla RMS) is sufficient to modulate vagus nerve activity. To ensure adequate nerve modulation, a RF magnetic flux density in the range of 0.5-5 microTesla peak, at the site of the vagus nerve, is required. These intensities are consistent with RF fields shown to be capable of modifying free radical pair recombination rates, which occur at RF frequencies ranging from 5-50 MHz (Henbest, et al., 2004; Wiltschko, et al., 2015), indicating that the effective RF frequency range for influencing the inflammatory response in animals would also be 5-50 MHz.

Shortwave radio frequency (RF) magnetic fields are created by passing an electric current through a “current loop”. This loop can be a single loop of wire or a coil of wire. The RF magnetic field created is perpendicular to the plane of the current loop. Correspondingly, there are different coil configurations that can be used to non-invasively expose the vagus nerve to RF magnetic fields. A single loop of wire creates a dipole magnetic field. For exposing the vagus nerve, such a loop could be positioned on the side or back of the neck of a human or other animal spanning the cervical vertebra of C1-C7. The magnetic field intensity within the tissue of the user will decrease inversely with distance from the current loop to the vagus nerve, such that, for a 6 cm radius loop centered on C3 at the back surface of a neck which is about 6-7 cm from the vagus nerve, the magnetic field flux density at the location of the vagus nerve will be approximately 50% of the flux density in the tissue at the back of the neck. An alternative coil configuration would be to form the wire loop into an elliptical configuration, and then conform the loop around the neck, so that the loop extends from the back, or front, of the neck along the sides of the neck, thereby positioning a portion of the current loop close to the vagus nerves. Another alternative current loop configuration would be to utilize a flexible wire that passes around the neck. This could be configured as a single loop or two or more loops. In this configuration, the wire loop would have to include a connector so that the loop could be opened up, placed around the neck, and then reconnected. Such a flexible wire loop would be expected to settle onto the shoulders, requiring a higher power to ensure adequate RF magnetic flux at the site of the vagus nerve, and so an alternative wire loop design would be to incorporate a loop tightening mechanism on the loop, allowing the user to effectively “shorten” the wire loop length in order to obtain a close fit to the neck. A fourth current loop configuration would be to utilize stretchable wire so that the current loop conformed to the shape of the neck. Stretchable wires are constructed by winding a fine wire around an elastic core. Stretchable wires can typically be expanded to 140% of their resting length. This would allow a single size elastic wire loop (for example, one of 30 cm in length) to be used on most women with neck diameter sizes ranging from 30 cm to 42 cm (i.e. 12 inches to 17 inches—the average woman neck size is 13.7 inches). Similarly, a single elastic wire loop of 38 cm could to be used by men with neck sizes of 38-53 cm (15 inches to 21 inches—the average neck size of a man is 16 inches). A fifth current loop configuration would be to include Flexible or Stretchable wire inside a cosmetic covering (fabric, band, etc.) to provide an aesthetically pleasing look to the device. An alternative to the current loop concept would be to utilize one or two (one on each side of the head) wound coils to expose the vagus nerve as it passes below the ear. There are distinct benefits to this configuration as the vagus nerve is closer to the skin at this location, and the specific site of the nerve is relatively easy to locate. Correspondingly, delivering a reproducible RF magnetic field intensity at this site becomes easier to implement than exposing the nerve lower on the neck region of the human or other animal.

While the experimental data demonstrated the ability of PSWT to produce an anti-inflammatory response in the limb joints, the vagus nerves modulate the activity of the systemic immune system and so the observed anti-inflammatory response can be expected to occur in any tissue which is experiencing chronic inflammation.

In summary, a wide variety of electrical and magnetic stimulation techniques have been developed for clinical applications of nerve stimulation and many of these have been applied to stimulation of the vagus nerve. However, in all vagal nerve studies, only brief stimulation durations have been implemented, as longer duration, high intensity electrical stimulation of the nerve has been found to have detrimental side effects. However, chronic inflammation is the predominant clinical challenge which needs to be addressed, and chronic conditions require a safe and effective, sustained, not intermittent, therapy.

Further, the present disclosure describes a vagal nerve stimulation (VNS) device capable of being used for extended time durations. Further, the VNS device may include multiple loops which encircles the neck of the user. Further, the VNS device may have a variety of different sized antenna loops for neck sizes ranging from 12″ to 25″. Further, the VNS device may have an adjustable antenna length for different sized necks. Further, the VNS device may include a variety of antenna loop connectors such as snap, press, loop, etc. Further, the VNS device may include a conducting band to better align the RF magnetic field with orientation of the vagus nerve in the neck. Further, the VNS device may generate higher power levels for deeper penetration (5-10 microTesla). Further, the VNS device allows changing RF magnetic field frequency, modulation frequency, and pulse widths. Further, the VNS device may look like a necklace. Further, the VNS device has an elastic package. Further, the VNS device may include a rechargeable battery, an on-off control mechanism, and an auto sensing mechanism. Further, the auto sensing mechanism is whether the VNS device is in a position on human or animal. Further, the VNS device may have a “patch” design and an ear-clip design. Further, the VNS device may facilitate 5G energy harvest for powering the VNS device. Further, the VNS device may include a nuclear battery and an internal heating coil to give a sense of perception while the device is on or even a vibration pulse for a sense of perception. Further, the VNS device may include interweaved graphene antenna insulation to increase the antenna strength from breaking. Further, the graphene enhanced antenna wire increases efficiency. Further, the VNS device may allow induction battery charging using a flat base station pad, with built in RF magnetic to align the antenna and keep the battery module flush with the base station pad. Further, the VNS device is water resistant or waterproof and may have no detectable ON/OFF switch. Further, the VNS device may include Wi-Fi or Bluetooth chip sets which include a rechargeable battery. Further, a setting may be downloaded from a cloud server via cell phone telemetry. Various software programs would need to be implemented for cellphone or PC telemetry. Further, the VNS device can be monitored and controlled for dosage.

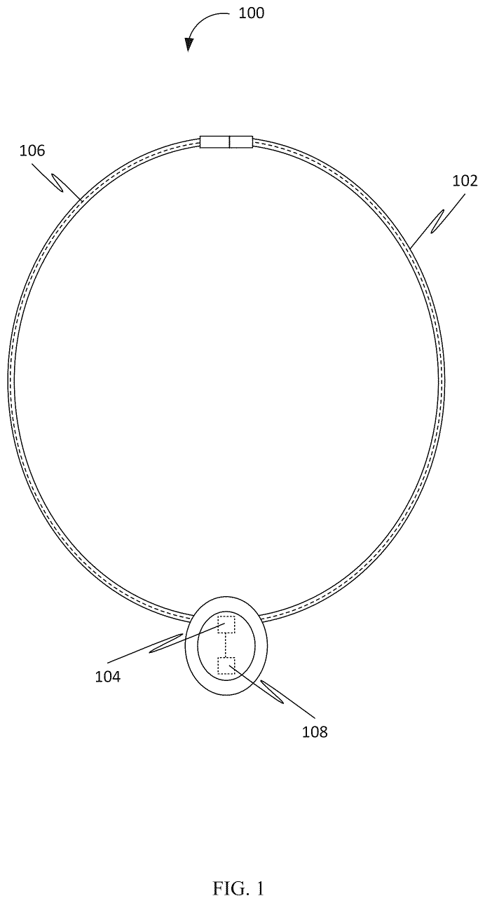

FIG. 1 is a front view of an apparatus 100 for facilitating stimulating of vagal nerves of a user, in accordance with some embodiments. Accordingly, the apparatus 100 may include an apparatus body 102, a signal generating device 104, at least one field generating element 106, and at least one powering device 108. Further, the apparatus 100 may be a VNS device.

Further, the apparatus body 102 may be configured to be placed on at least one part of a body of the user. Further, the at least one part of the body may be associated with at least one nerve. Further, the at least one vagal nerve may be a branch of the vagus nerve. Further, the at least one part of the body may include a neck, a head, etc. Further, the user may include a human, an animal, etc. Further, the placing of the apparatus body 102 on the at least one part of the body of the user may include placing the apparatus body at the site of the at least one vagal nerve.

Further, the signal generating device 104 may be placed in the apparatus body 102. Further, the signal generating device 104 may be configured for generating at least one signal with at least one signal characteristic. Further, the at least one signal generating device 104 may include an electromagnetic device, an oscillator, a signal generator, a function generator, etc. Further, the at least one signal may include an electrical signal (such as a current signal, a voltage signal, etc.). Further, the at least one signal characteristic may be a specific signal characteristic. Further, the at least one signal characteristic may include a specific amplitude, a specific carrier frequency, a specific pulse width, a specific waveform, a specific modulation frequency, etc. Further, the specific waveform may include a square waveform. Further, the specific carrier frequency may include 6.8 MHz, 13.5 MHz, 40.7 MHz, 43.4 MHz, 27.1 MHz, etc. Further, the specific modulation frequency may include 0.1 kHz, 0.5 kHz, 1 kHz, 2 kHz, 4 kHz, 8 kHz, etc. Further, the specific pulse width may include 100 microseconds, 200-1000 microseconds, etc.

Further, the at least one field generating element 106 may be placed in the apparatus body 102. Further, the at least one field generating element 106 may be coupled with the signal generating device 104. Further, the at least one field generating element 106 may be electrically coupled with the signal generating device 104. Further, the at least one field generating element 106 may be configured for generating at least one RF magnetic field with at least one RF magnetic field characteristic based on the generating of the at least one signal. Further, the at least one field generating element 106 may include a conducting wire, a conducting strip, a conducting band, a coil, etc. Further, the at least one field generating element 106 may include a single conductor, a plurality of conductors, etc. Further, the single conductor, the plurality of conductors, etc. of the at least one field generating element 106 may be a transducer. Further, the transducer may output an electromagnetic RF field. Further, the single conductor, the plurality of conductors, etc. may be associated with a strap, an enclosure, etc. Further, the strap, the enclosure, etc. may cover the single conductor, the plurality of conductors, etc. Further, the strap, the enclosure, etc. may be a type of insulator that engulfs the single conductor, the plurality of conductors, etc. Further, the at least one field generating element 106 may be an insulated wire. Further, the at least one RF magnetic field characteristic may correspond to the at least one signal characteristic. Further, the at least one RF magnetic field characteristic may be a specific RF magnetic field characteristic. Further, the at least one RF magnetic field characteristic may include a specific strength, a specific direction, a specific shape, a specific symmetry, a specific loop area, a specific flux density, a specific frequency, a specific pulse width, a specific pulse rate, a specific pulse duty cycle, etc. Further, the specific flux density may include 1 microTesla, <1 microTesla, 1-5 microTesla, 5-10 microTesla, 0.5 microTesla, 0.5-5 microTesla, 0.35 microTesla, 0.1-100 microTesla, etc. Further, the specific flux density may be a specific peak value, a specific RMS value, etc. Further, the specific frequency may include 5-50 MHz, 27.1 MHz, etc. Further, the specific pulse rate may include 100-10,000 Hz, 1000 Hz, etc. Further, the specific pulse duty cycle may include 10%-50%, 10%, etc. Further, the at least one RF magnetic field with the at least one RF magnetic field characteristic may be applied to the at least one vagal nerve based on the placing of the apparatus body 102 and the generating of the at least one RF magnetic field with the at least one RF magnetic field characteristic. Further, the application of the at least one RF magnetic field with the at least one RF magnetic field characteristic may be associated with at least one time period and at least one course for suppressing a chronic inflammatory response in the body of the user. Further, the at least one time period may be an extended time period.

Further, the at least one powering device 108 may be placed in the apparatus body 102. Further, the at least one powering device 108 may be electrically coupled with the signal generating device 104. Further, the at least one powering device 108 may be configured for powering the signal generating device 104. Further, the generating of the at least one signal with the at least one signal characteristic may be based on the powering. Further, the at least one powering device 108 may include a battery, a nuclear battery, a capacitor, etc.

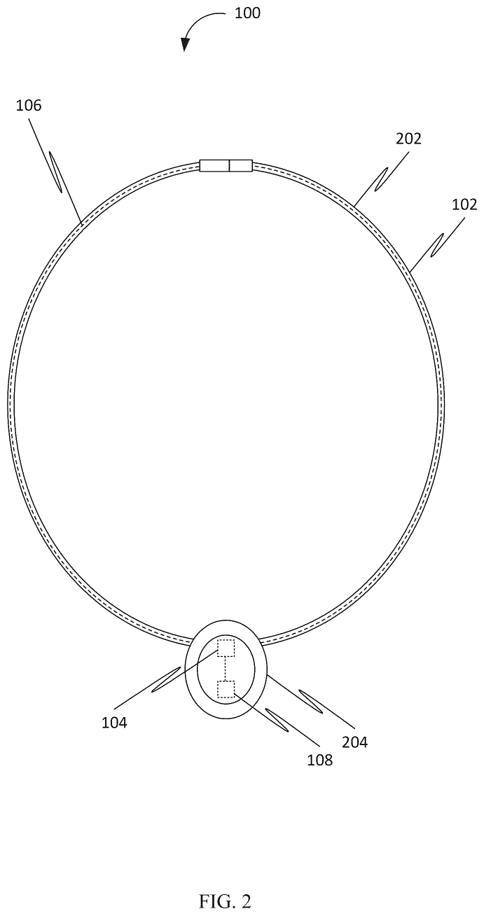

FIG. 2 is a front view of the apparatus 100 for facilitating the stimulating of the vagal nerves of the user, in accordance with some embodiments. Accordingly, the apparatus body 102 may include a neck strap portion 202 and a pendant portion 204 attached to the neck strap portion 202. Further, the apparatus body 102 may be shaped as a necklace. Further, the at least one part of the body may include a neck of the user. Further, the neck strap portion 202 may be placed around the neck of the user. Further, the at least one field generating element 106 may be comprised in the neck strap portion 202. Further, the signal generating device 104 and the at least one powering device 108 may be comprised in the pendant portion 204. Further, the neck strap portion 202 may include a strap. Further, the pendant portion 204 may include a closed case with a cavity to hold one or more devices. Further, the neck strap portion 202 encloses the at least one field generating element 106. Further, the neck strap portion 202 may be an insulator coating for the at least one field generating element 106.

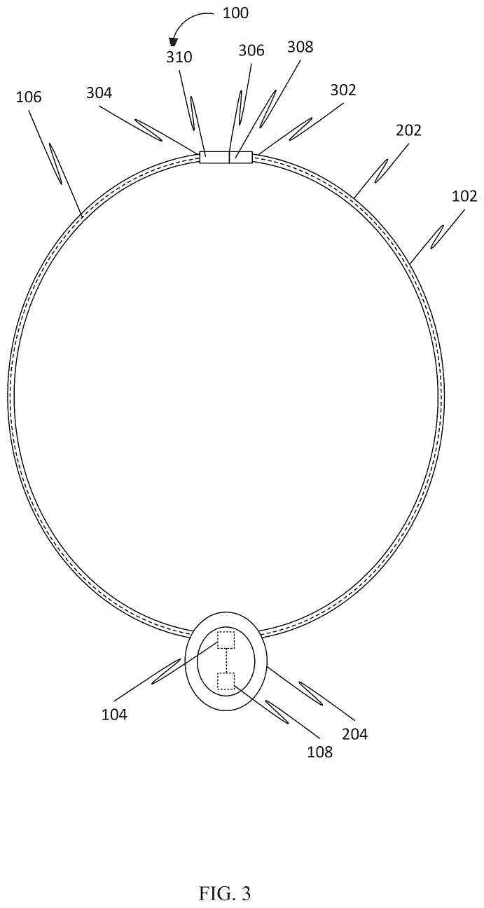

FIG. 3 is a front view of the apparatus 100 for facilitating the stimulating of the vagal nerves of the user, in accordance with some embodiments. Accordingly, the neck strap portion 202 extends between a first end 302 and a second end 304. Further, the neck strap portion 202 may include a connecting element 306. Further, the connecting element 306 may be configured for transitioning a loop formed by the neck strap portion 202 between a continuous loop and a discontinuous loop. Further, the connecting element 306 may include a first connecting portion 308 and a second connecting portion 310. Further, the first connecting portion 308 may be comprised in the first end 302 of the neck strap portion 202 and the second connecting portion 310 may be comprised in the second end 304 of the neck strap portion 202. Further, the first connecting portion 308 may be disconnectably connectable to the second connecting portion 310 for the transitioning of the neck strap portion 202 between the continuous loop and the discontinuous loop. Further, the connecting element 306 may include a snap connector, a latch, etc.

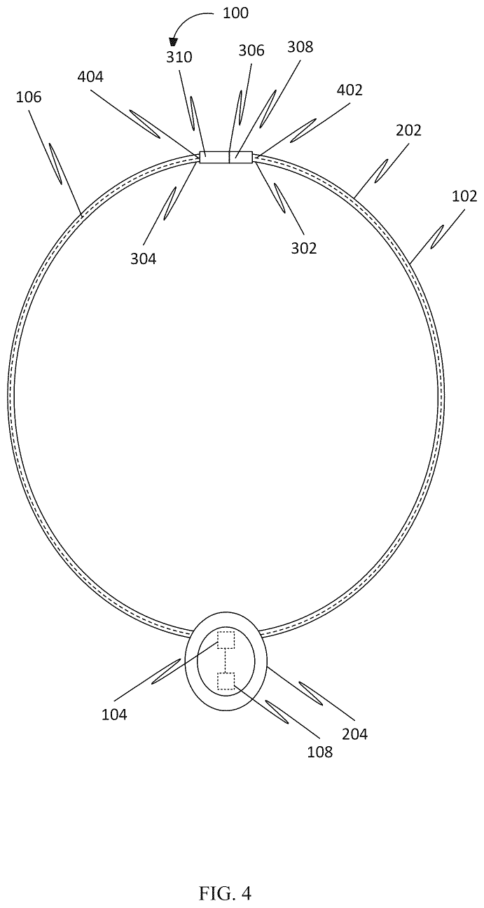

FIG. 4 is a front view of the apparatus 100 for facilitating the stimulating of the vagal nerves of the user, in accordance with some embodiments. Accordingly, the at least one field generating element 106 extends between a first end 402 and a second end 404. Further, the first end 402 of the at least one field generating element 106 may be comprised in the first end 302 of the neck strap portion 202 and the second end 404 of the at least one field generating element 106 may be comprised in the second end 304 of the neck strap portion 202. Further, the transitioning of the loop formed by the neck strap portion 202 between the continuous loop and the discontinuous loop transitions the loop formed by the at least one field generating element 106 between the continuous loop and the discontinuous loop. Further, the generating of the at least one RF magnetic field with the at least one RF magnetic field characteristic may be further based on the transitioning of the loop formed by the at least one field generating element 106 to the continuous loop.

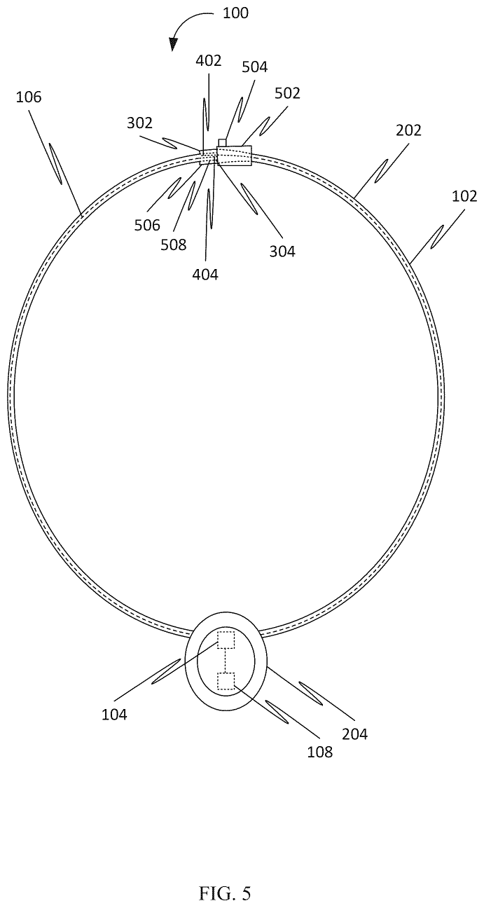

FIG. 5 is a front view of the apparatus 100 for facilitating the stimulating of the vagal nerves of the user, in accordance with some embodiments. Accordingly, the at least one field generating element 106 extends between the first end 402 and the second end 404. Further, the first end 402 of the at least one field generating element 106 may be comprised in the first end 302 of the neck strap portion 202 and the second end 404 of the at least one field generating element 106 may be comprised in the second end 304 of the neck strap portion 202. Further, the neck strap portion 202 may include a loop tightening mechanism 502 coupled to the first end 302 of the neck strap portion 202 and the second end 304 of the neck strap portion 202. Further, the loop tightening mechanism 502 may be configured for modifying a length of the loop of the neck strap portion 202 and the at least one field generating element 106 based on at least one action receivable by a tightening element 504 of the loop tightening mechanism 502. Further, the modifying of the length may include connectedly overlapping at least one length of at least one of a first portion 506 of the neck strap portion 202 and the at least one field generating element 106 over a second portion 508 of the neck strap portion 202 and the at least one field generating element 106. Further, the loop tightening mechanism 502 may include a rotary dial, a latch, etc.

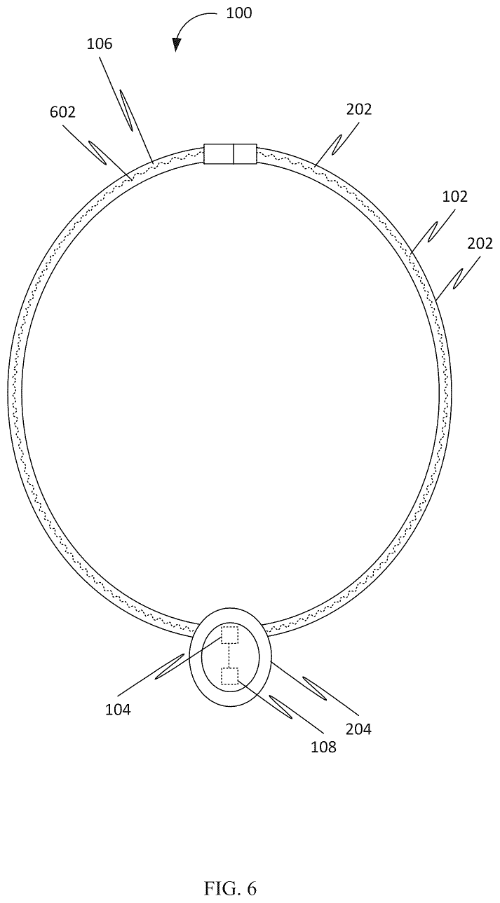

FIG. 6 is a front view of the apparatus 100 for facilitating the stimulating of the vagal nerves of the user, in accordance with some embodiments. Accordingly, the neck strap portion 202 may be comprised of at least one elastic material. Further, the at least one field generating element 106 may include a filament 602. Further, the filament 602 may be arranged in at least one configuration. Further, the at least one configuration may include a shape of the filament 602. Further, the at least one configuration may include may include a weaved configuration, a coiled configuration, etc. Further, the neck strap portion 202 and the at least one field generating element 106 may be configured to be transitioned from an original state to at least extended state by applying at least one external force on the neck strap portion 202 based on the at least one elastic material and the at least one configuration of the filament 602. Further, the neck strap portion 202 and the at least one field generating element 106 may be transitionable from the at least one extended state to the original state by removing the at least one external force from the neck strap portion 202 based on the at least one elastic material and the at least one configuration of the filament 602. Further, the at least one elastic material may include rubber, hard rubber, interweaved graphene, etc. Further, the filament 602 may include a conducting wire wound on the at least one elastic material.

Further, in some embodiments, the at least one field generating element 106 comprised of at least one electrically conducting material. Further, the at least one field generating element 106 may be characterized by a length and a cross sectional area. Further, the generating of the at least one RF magnetic field with the at least one RF magnetic field characteristic may be further based on the at least one electrically conducting material, the length, and the cross sectional area of the at least one field generating element 106. Further, the at least one electrically conducting material may include copper, iron, silver, graphene, etc.

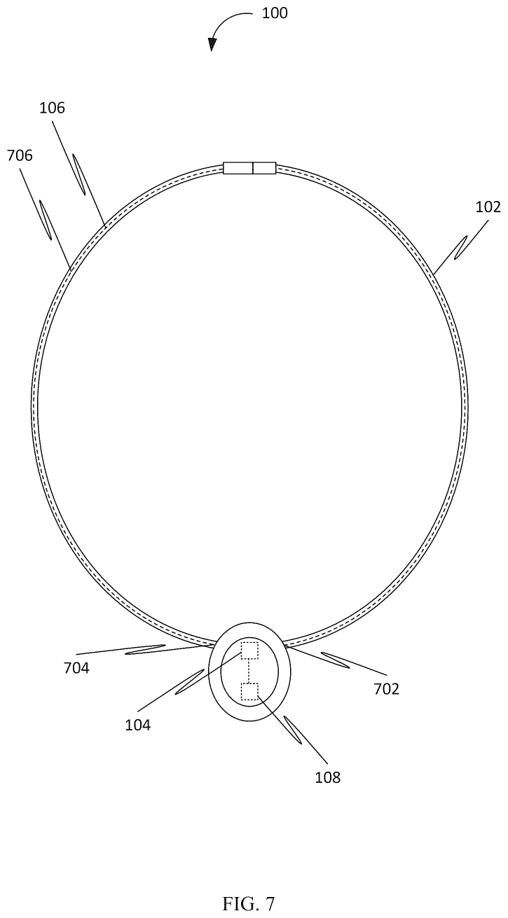

FIG. 7 is a front view of the apparatus 100 for facilitating the stimulating of the vagal nerves of the user, in accordance with some embodiments. Accordingly, the at least one field generating element 106 extends between a first end 702 and a second end 704. Further, the first end 702 of the at least one field generating element 106 and the second end 704 of the at least one field generating element 106 may be coupled to the signal generating device 104 for forming at least one loop 706. Further, the generating of the at least one RF magnetic field with the at least one RF magnetic field characteristic may be based on the forming of the at least one loop 706.

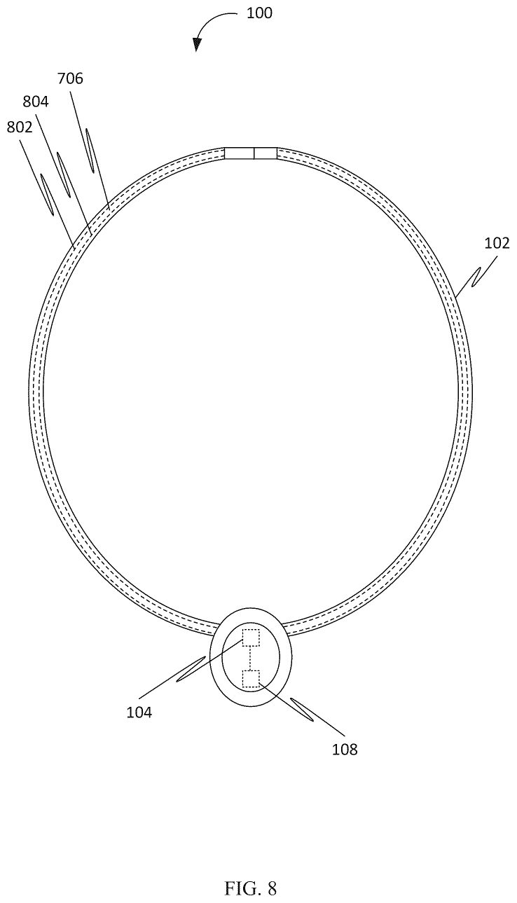

FIG. 8 is a front view of the apparatus 100 for facilitating the stimulating of the vagal nerves of the user, in accordance with some embodiments. Accordingly, the at least one loop 706 may include a plurality of loops 802-804. Further, the plurality of loops 802-804 may be interconnected.

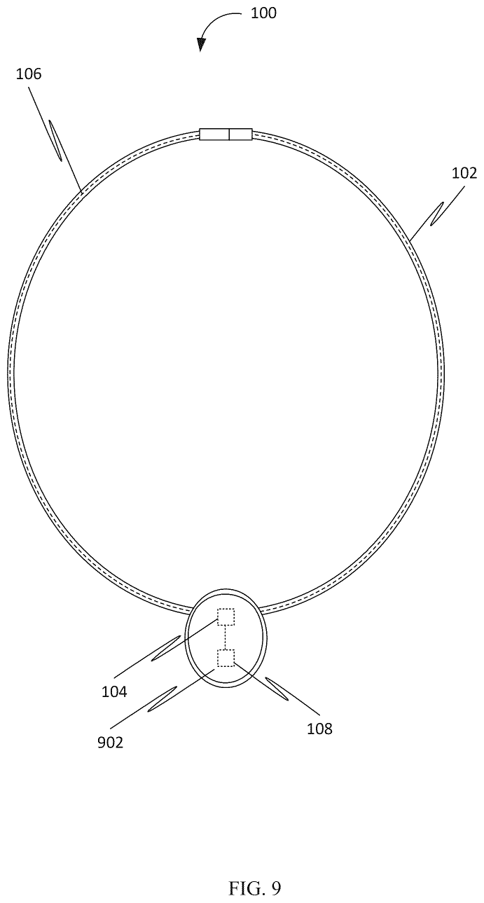

FIG. 9 is a rear view of the apparatus 100 for facilitating the stimulating of the vagal nerves of the user, in accordance with some embodiments. Accordingly, the apparatus body 102 may be further configured to be attached to a surface of the at least one part of the body of the user using at least one attaching element 902. Further, the surface may be a skin of the body. Further, the disposing of the apparatus body 102 on the at least one part of the body may be based on the attaching of the apparatus body 102 to the at least one part of the body. Further, the at least one attaching element 902 may include an adhesive sheet.

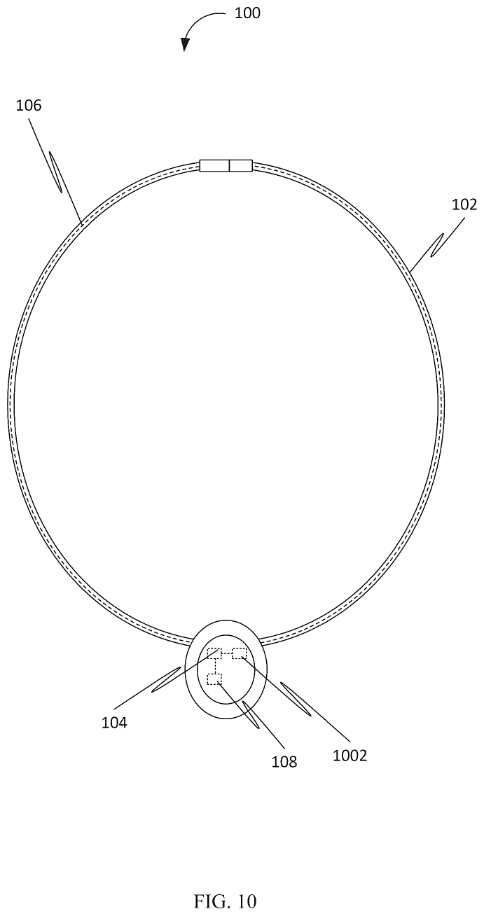

FIG. 10 is a rear view of the apparatus 100 for facilitating the stimulating of the vagal nerves of the user, in accordance with some embodiments. Accordingly, the apparatus 100 may include a processing device 1002. Further, the processing device 1002 may be comprised in the apparatus body 102. Further, the processing device 1002 may be configured for analyzing at least one data. Further, the at least one data may include at least one time period data associated with the at least one time period and at least one course data associated with the at least one course. Further, the at least one course may include a therapy course. Further, the at least one time period may include 12-24 hours per day. Further, the at least one course may include 7-14 days. Further, the at least one data may include a dosage information associated with a dosage of the at least one RF magnetic field, a schedule for exposing the at least one body part to the at least one RF magnetic field, a requirement of the at least one RF magnetic field characteristic of the at least one RF magnetic field, etc. Further, the processing device 1002 may be configured for determining at least one value for at least one parameter associated with the generating of the at least one signal with the at least one signal characteristic based on the analyzing of the at least one data. Further, the at least one parameter may include an activation time, an activation duration, a number of pulses, a frequency of pulses, a strength of RF magnetic field generated by each pulse, a flux density of RF magnetic field generated by each pulse, etc. Further, the at least one parameter may include an amplitude, a waveform, a frequency, a pulse width, a duty cycle, a modulation, an output impedance, an attenuation, etc. Further, the processing device 1002 may be configured for generating at least one command for the signal generating device 104 based on the determining. Further, the signal generating device 104 may be operatively coupled with the processing device 1002. Further, the generating of the at least one signal with the at least one characteristic may be based on the at least one command.

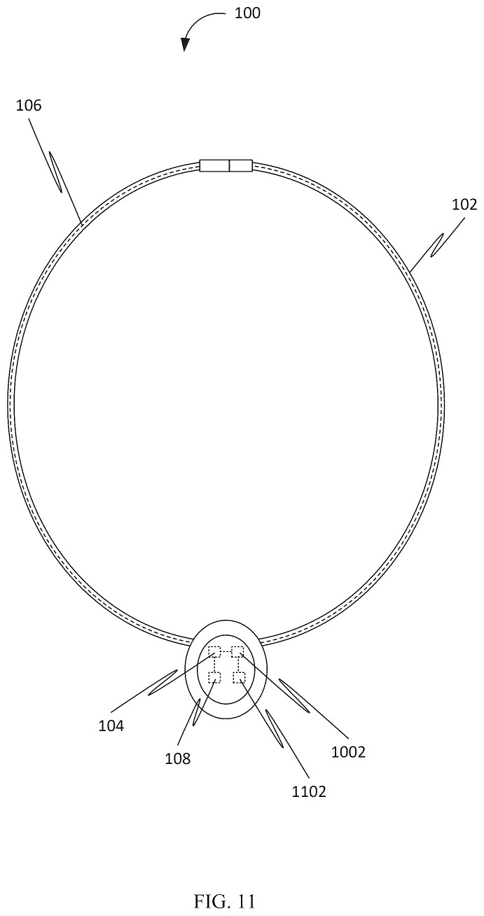

FIG. 11 is a front view of the apparatus 100 for facilitating the stimulating of the vagal nerves of the user, in accordance with some embodiments. Accordingly, the apparatus 100 may include at least one communication interface 1102 comprised in the apparatus body 102. Further, the at least one communication interface 1102 may be communicatively coupled with the processing device 1002. Further, the at least one communication interface 1102 may be configured for receiving the at least one data from at least one device. Further, the at least one device may include a computing device, a client device, an IoT device, etc. Further, the at least one communication interface may include a Bluetooth chipset, a Wi-Fi chipset, etc.

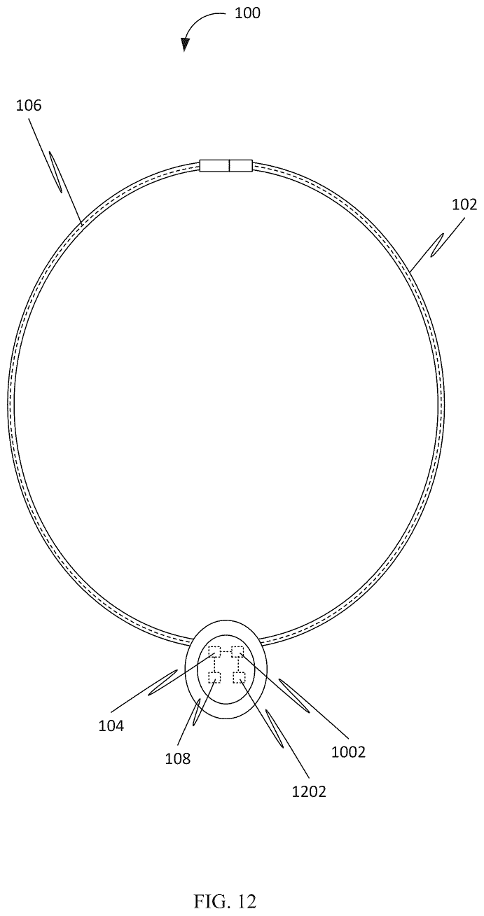

FIG. 12 is a front view of the apparatus 100 for facilitating the stimulating of the vagal nerves of the user, in accordance with some embodiments. Accordingly, the apparatus 100 may include at least one sensor 1202 comprised in the apparatus body 102. Further, the at least one sensor 1202 may be communicatively coupled with the processing device 1002. Further, the at least one sensor 1202 may include an X ray transmitter and an X ray receiver for imaging the at least one part of the body. Further, the at least one sensor 1102 may be configured for imaging the at least one field generating element 106 and the at least one vagal nerve. Further, the processing device 1002 may be further configured for generating an imaging data based on the imaging. Further, the at least one sensor may include an ultrasonic sensor, a radio sensor, an infrared sensor, etc. Further, the processing device 1002 may be configured for analyzing the imaging data. Further, the processing device 1002 may be configured for determining a proximity between the at least one field generating element 106 and the at least one vagal nerve based on the analyzing of the imaging data. Further, the determining of the at least one value for the at least one parameter may be further based on the analyzing of the proximity data.

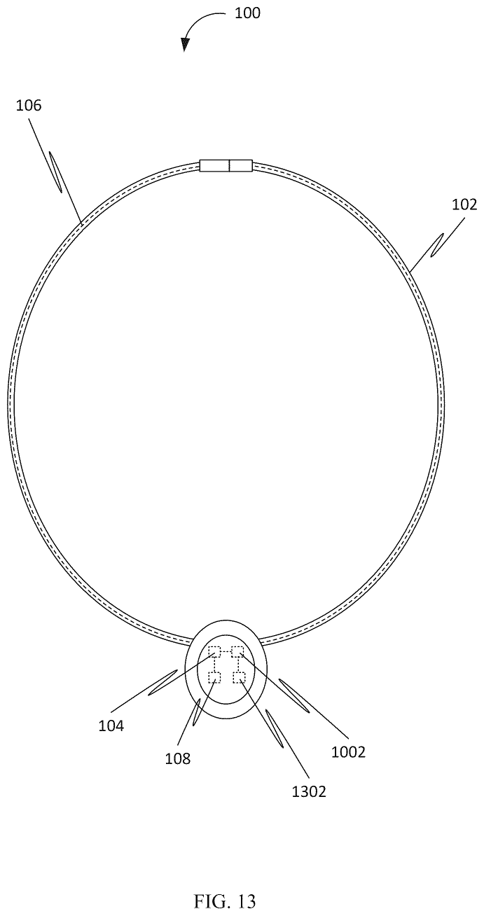

FIG. 13 is a front view of the apparatus 100 for facilitating the stimulating of the vagal nerves of the user, in accordance with some embodiments. Accordingly, the apparatus 100 may include at least one biological sensor 1302 comprised in the apparatus body 102. Further, the at least one biological sensor 1302 may be communicatively coupled with the processing device 1002. Further, the at least one biological sensor 1302 may be configured for detecting a characteristic of the at least one part of the body. Further, the at least one biological sensor 1302 may include a bioimpedance sensor, a near-infrared spectroscopy (NIRS) sensor, an ultrasound sensor, a magnetic resonance spectroscopy (MRS) sensor, an optical coherence tomography (OCT) sensor, etc. Further, the characteristic may include a tissue physical composition, a tissue structure, a tissue chemical composition, a tissue physical property (such as density, elasticity, etc.), a parameter (such as tissue conductivity, cellularity, fluid distribution, tissue oxygenation, blood flow, fat content, etc.), etc. Further, the processing device 1002 may be further configured for generating a biological data based on the detecting of the characteristic. Further, the processing device 1002 may be configured for analyzing the biological data. Further, the biological data may be analyzed to determine a magnetic permeability of the at least one part of the body, an electrical tolerance of the at least one part of the body, etc. Further, the determining of the at least one value for the at least one parameter may be based on the analyzing of the biological data.

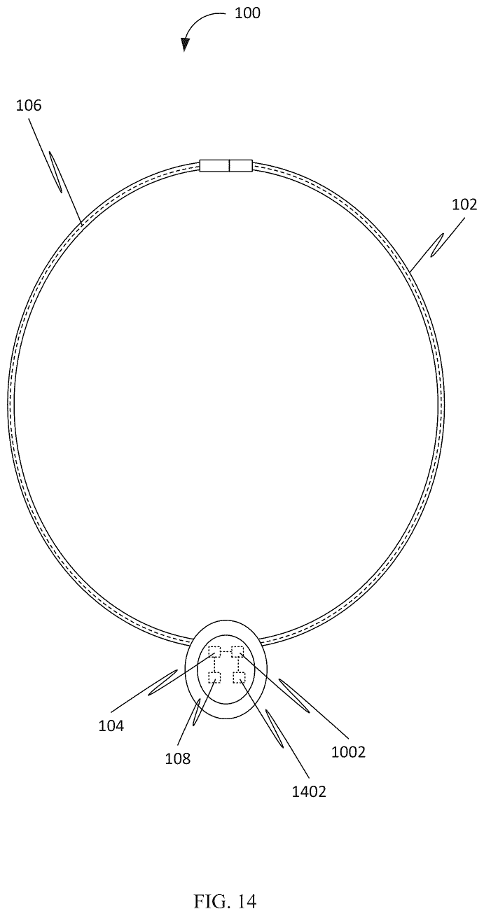

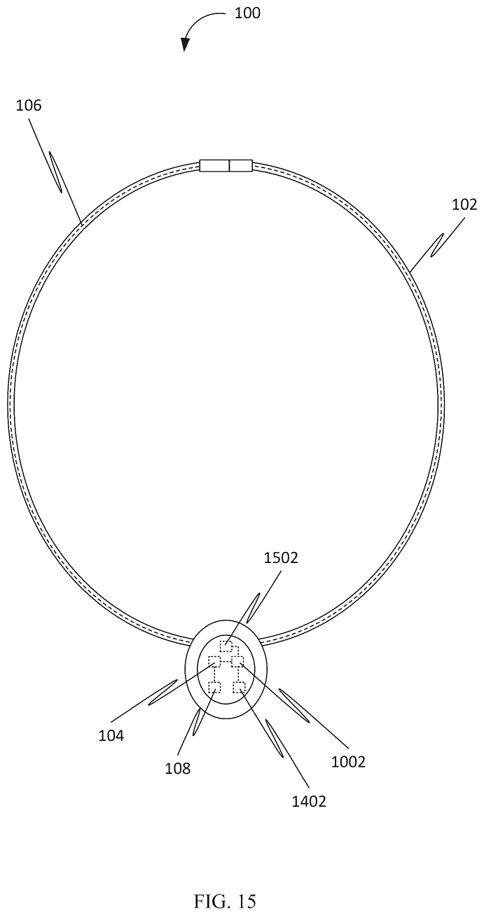

FIG. 14 is a front view of the apparatus 100 for facilitating the stimulating of the vagal nerves of the user, in accordance with some embodiments. Accordingly, the apparatus 100 may include at least one position sensor 1402 comprised in the apparatus body 102. Further, the at least one position sensor 1402 may be communicatively coupled with the processing device 1002. Further, the at least one position sensor 1402 may be configured for detecting a position of the at least one field generating element 106 in relation to the at least one vagal nerve. Further, the processing device 1002 may be communicatively coupled with the at least one position sensor 1402. Further, the processing device 1002 may be configured for generating a position data based on the detecting of the position. Further, the processing device 1002 may be configured for analyzing the position data based on at least one criterion. Further, the at least one criterion may include a position with respect to a C1 vertebra of the body. Further, the processing device 1002 may be configured for determining an alignment of the at least one field generating element 106 in relation to the at least one vagal nerve based on the analyzing of the position data. Further, the processing device 1002 may be configured for generating an output signal corresponding to the alignment based on the determining. Further, the apparatus 100 may include at least one output device 1502, as shown in FIG. 15. Further, the at least one output device 1502 may be comprised in the apparatus body 102. Further, the at least one output device 1502 may be communicatively coupled with the processing device 1002. Further, the at least one output device 1502 may be configured for generating at least one output based on the output signal. Further, the at least one output device 1502 may include a speaker, a lighting device, etc. Further, the at least one output may include one or more sounds, one or more lights, etc.

FIG. 15 is a front view of the apparatus 100 for facilitating the stimulating of the vagal nerves of the user, in accordance with some embodiments.

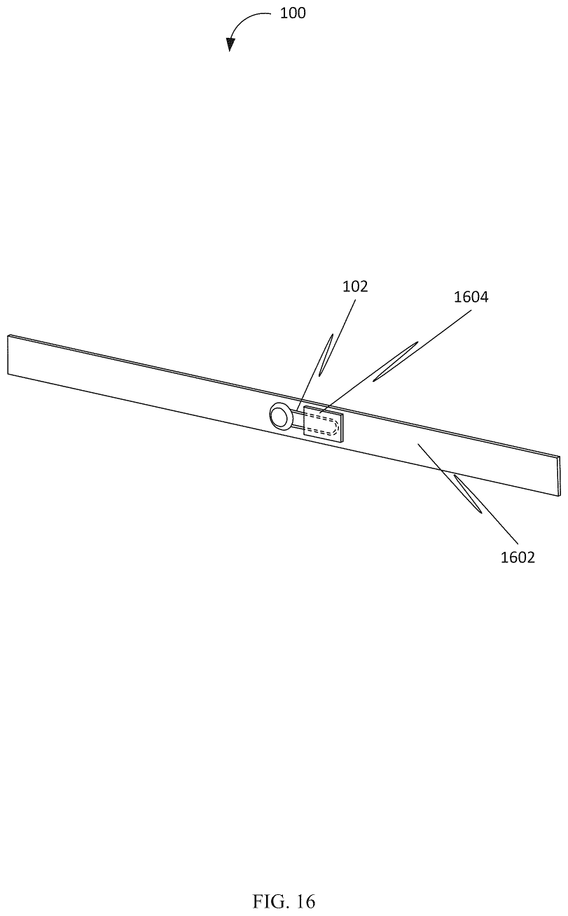

FIG. 16 is a perspective view of the apparatus 100 for facilitating the stimulating of the vagal nerves of the user, in accordance with some embodiments. Accordingly, the apparatus 100 may include a wearable apparel 1602 configured to be worn on the at least one part of the body. Further, the wearable apparel 1602 may include a collar, a strap, etc. Further, the wearable apparel 1602 may include at least one holder 1604. Further, the at least one holder 1604 may be configured for securing the apparatus body 102 in at least one location on the wearable apparel 1602. Further, the disposing of the body 102 on the at least one part of the body may be further based on the securing of the apparatus body 102 in the at least one location and the wearing of the wearable apparel 1602. Further, the wearable apparel 1602 may include a strap, a band, etc. Further, the at least one holder 1604 may include a pocket, a space, etc.

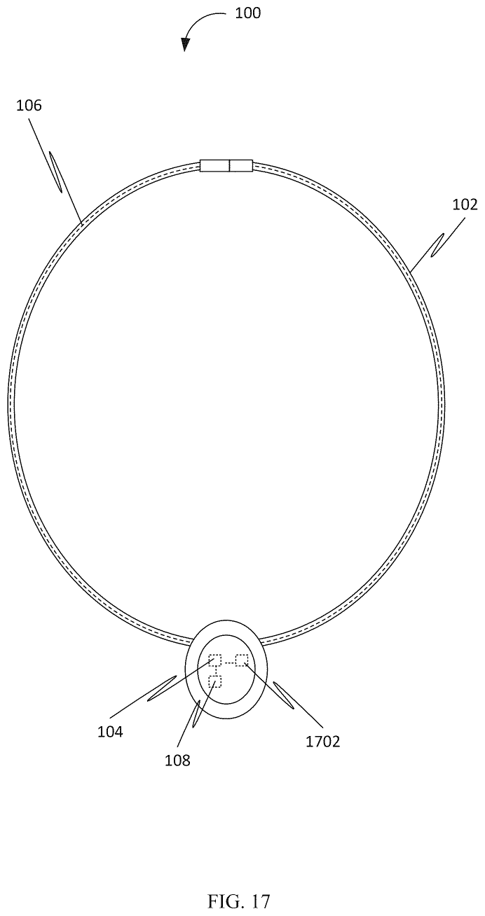

FIG. 17 is a front view of the apparatus 100 for facilitating the stimulating of the vagal nerves of the user, in accordance with some embodiments. Accordingly, the apparatus 100 may include at least one output device 1702 placed in the apparatus body 102. Further, the at least one output device 1702 may be operatively coupled with the signal generating device 104. Further, the at least one output device 1702 may be configured for outputting at least one stimulus perceptible to the user based on the generating of the at least one signal with the at least one signal characteristic. Further, the at least one output device 1702 may include a vibration generating device, a heat generating device, etc. Further, the at least one stimulus may include a vibration, a heating sensation, etc.

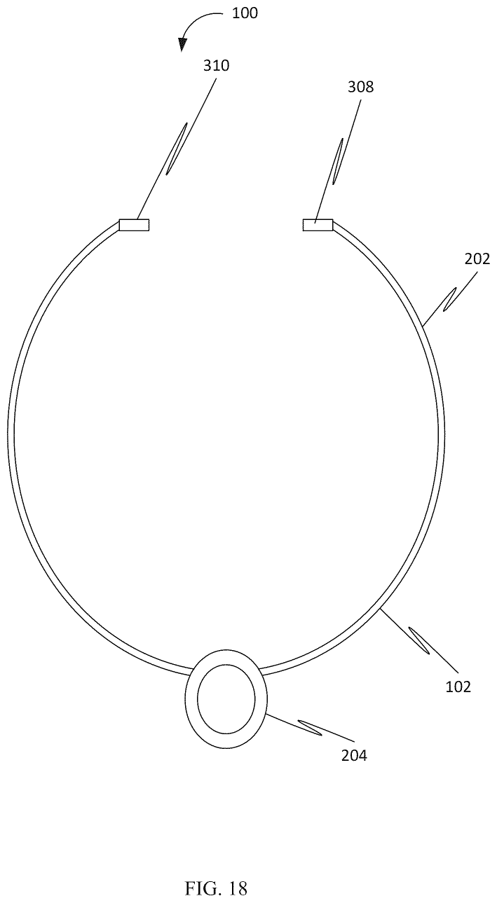

FIG. 18 is a front view of the apparatus 100 for facilitating the stimulating of the vagal nerves of the user, in accordance with some embodiments. Further, the loop formed by the loop formed by the neck strap portion 202 may be the discontinuous loop.

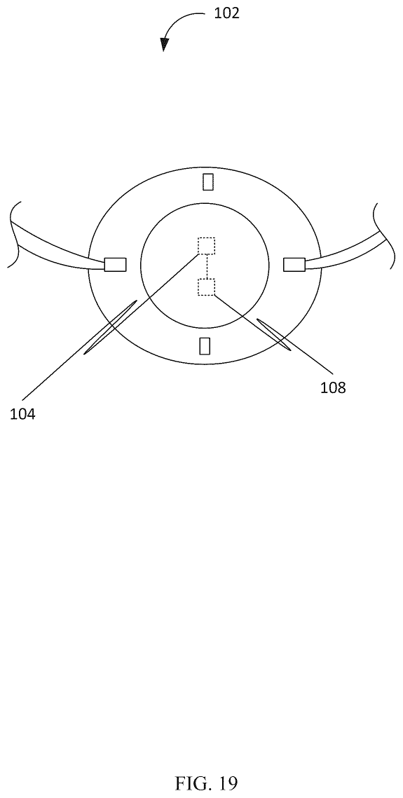

FIG. 19 is a partial rear view of the apparatus body 102 for facilitating the stimulating of the vagal nerves of the user, in accordance with some embodiments.

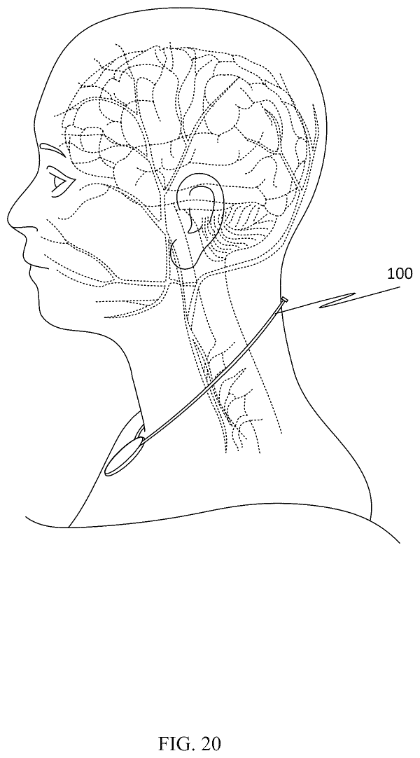

FIG. 20 is a side view of the apparatus 100 worn by the user, in accordance with some embodiments. Further, the user may be a human.

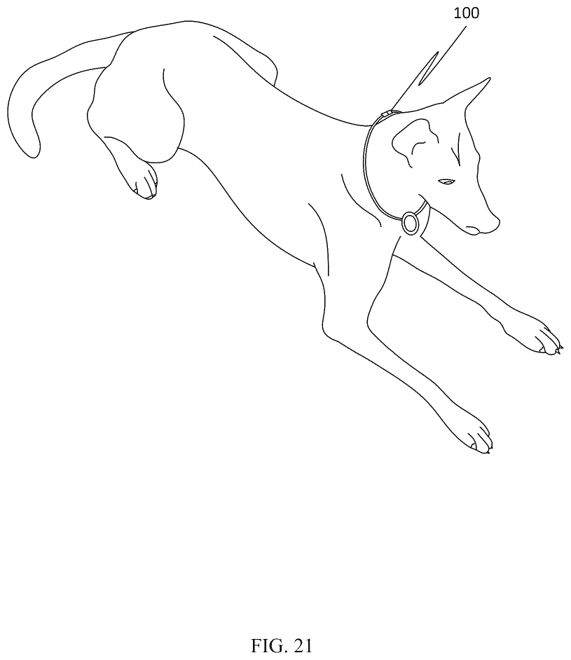

FIG. 21 is a side view of the apparatus 100 worn by the user, in accordance with some embodiments. Further, the user may be an animal.

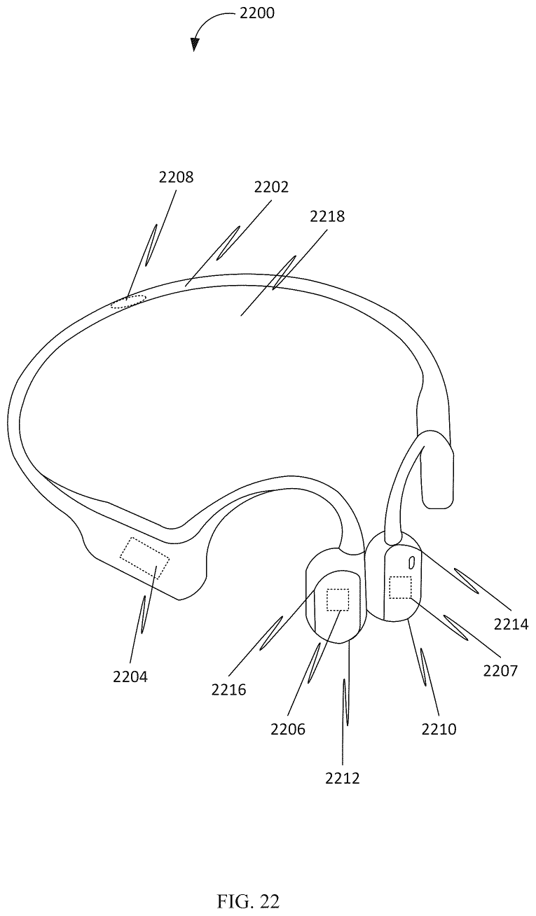

FIG. 22 is a perspective view of an apparatus 2200 for facilitating stimulating of the vagal nerves of the user, in accordance with some embodiments. Accordingly, the apparatus 2200 may include an apparatus body 2202, a signal generating device 2204, at least one field generating element 2206-2207, and at least one powering device 2208.

Further, the apparatus body 2202 may extends between a first end 2210 and a second end 2212. Further, the apparatus body 2202 may be curvedly shaped with a curvature for defining an interior space 2218. Further, the apparatus body 2202 may include a first body portion 2214 comprising the first end 2210 of the body 2202 and a second body portion 2216 comprising the second end 2212 of the body 2202. Further, the first body portion 2214 opposes the second body portion 2216. Further, the at least one part of the body may include a head of the user. Further, at least one of the first body portion 2214 and the second body portion 2216 may be transitionable from an original state to at least one extended state for transitioning the curvature from an original curvature to at least one extended curvature based on an application of at least one external force. Further, the apparatus body 2202 receives at least a portion of the head of the user in the interior space 2218 based on the transitioning of the curvature to the at least one extended curvature. Further, the first body portion 2214 and the second body portion 2216 transition to at least one retracted state from the at least one extended state for transitioning the curvature from the at least one extended curvature to at least one retracted curvature based on a removal of the at least one external force. Further, the first body portion 2214 and the second body portion 2216 presses against at least the portion of the head in the at least one retracted state for securing the apparatus body 2202 to the head. Further, the disposing of the apparatus body 2202 to the at least one part of the body may be further based on the securing of the first end 2210 and the second end 2212.

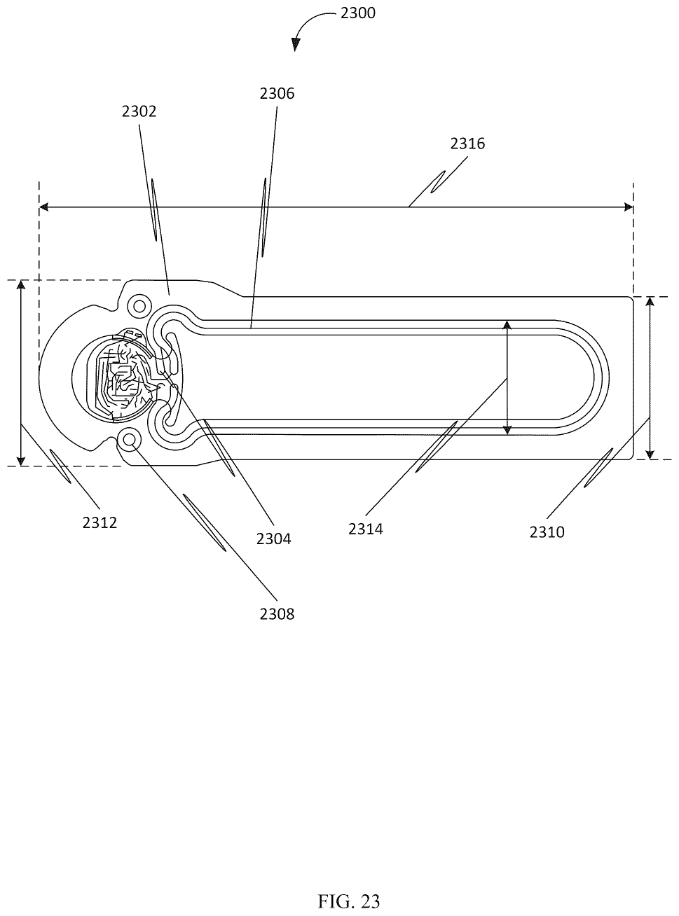

FIG. 23 is a front view of an apparatus 2300 for facilitating stimulating of vagal nerves of a user, in accordance with some embodiments. Accordingly, the apparatus 2300 may include an enclosure 2302, an ASIC chip 2304, an antenna 2306, and a powering device 2308.

Further, a width 2310 of the enclosure 2302 may be about 2.5 cm (˜1 inch) and a maximum width 2312 of the enclosure 2302 may be about 5 cm (˜2 inch). Further, a length 2316 of the enclosure 2302 may be about 30 cm (˜12 inch). Further, the enclosure 2302 may be an apparatus body of the apparatus 2300. Further, the enclosure 2302 includes the antenna 2306, the ASIC chip 2304, and the powering device 2308.

Further, the ASIC chip 2304 may be configured for generating at least one signal for generating at least one RF magnetic field. Further, the ASIC chip 2304 may be at least one signal generating device of the apparatus 2300.

Further, the antenna 2306 may be placed in the enclosure 2302. Further, the antenna 2306 may be configured for generating the at least one RF magnetic field based on the generating of the at least one signal. Further, the antenna 2306 may be at least one field generating element of the apparatus 2300. Further, a width 2314 of the antenna 2306 may be about 3 cm (1.2 inch).

Further, the powering device 2308 may be placed on the enclosure 2302. Further, the powering device 2308 may be configure to power the ASIC chip 2304.

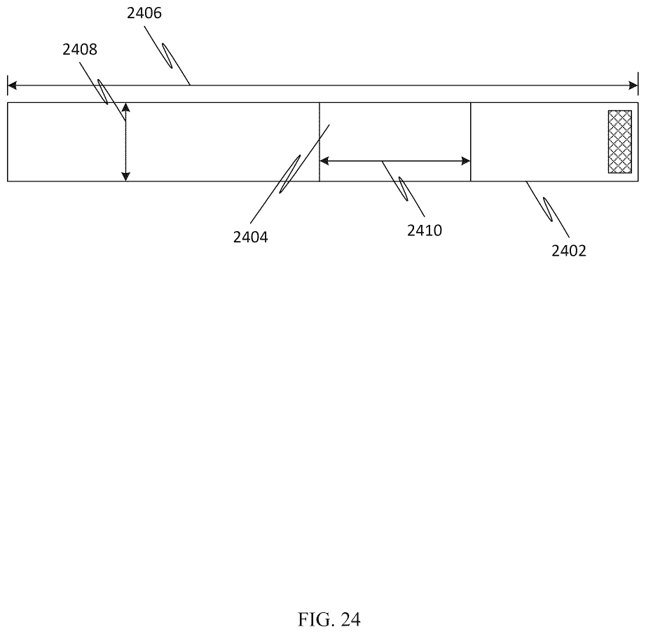

FIG. 24 is a front view of a strap 2402 for receiving an apparatus for facilitating stimulating of vagal nerves of a user, in accordance with some embodiments. Accordingly, the apparatus may be placed in a pocket 2404 of the strap 2402. Further, a length 2406 of the strap 2402 may be about 21 inches and a width 2408 of the strap 2402 may be about 2.5 inches. Further, a length 2410 of the pocket 2404 may be about 6 inches to enclose the enclosure 2302.

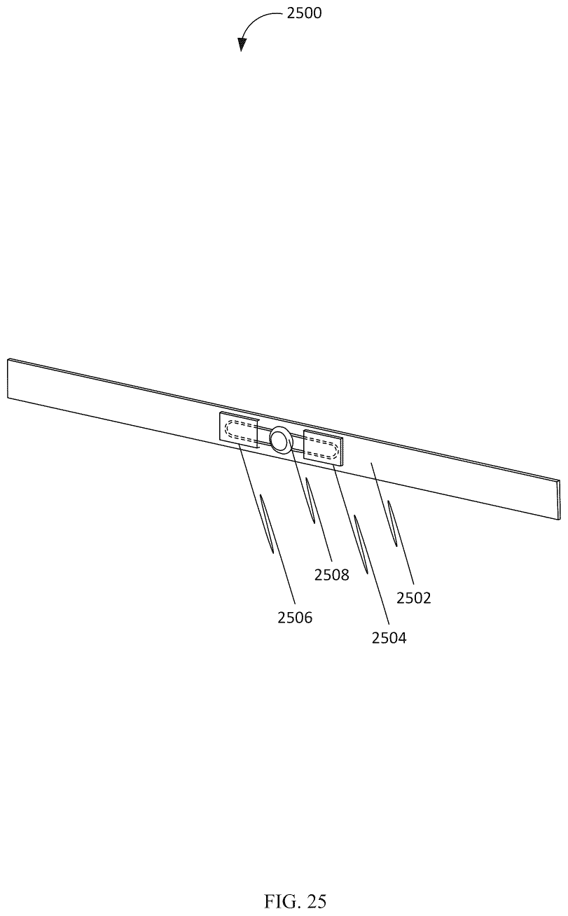

FIG. 25 is a perspective view of an apparatus 2500 for facilitating stimulating of vagal nerves of a user, in accordance with some embodiments. Accordingly, the apparatus 2500 may include a strap 2502 and an apparatus body 2504. Further, the strap 2502 may include a collar (animal collar). Further, the strap 2502 may include at least one enclosure 2506-2508. Further, the at least one enclosure 2506-2508 may be configured for receiving the apparatus body 2504.

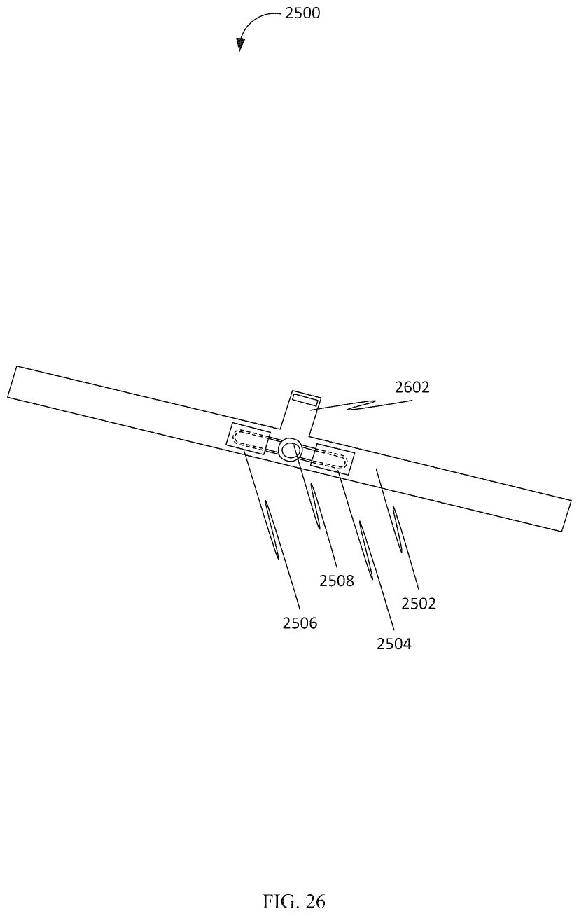

FIG. 26 is a perspective view of the apparatus 2500, in accordance with some embodiments. Further, the strap 2502 may include a harness holder 2602.

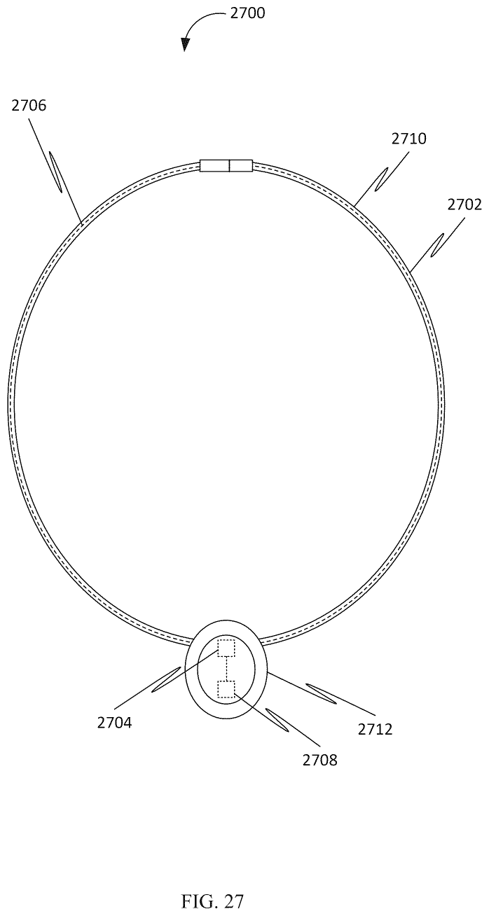

FIG. 27 is a front view of an apparatus 2700 for facilitating stimulating of vagal nerves of a user, in accordance with some embodiments. Accordingly, the apparatus 2700 may include an apparatus body 2702, a signal generating device 2704, at least one field generating element 2706, and at least one powering device 2708.

Further, the apparatus body 2702 may be configured to be placed on at least one part of a body of the user. Further, the at least one part of the body may be associated with at least one vagal nerve. Further, the apparatus body 2702 may include a neck strap portion 2710 and a pendant portion 2712 attached to the neck strap portion 2710. Further, the at least one part of the body may include a neck of the user. Further, the neck strap portion 2710 may be placed around the neck of the user.

Further, the signal generating device 2704 may be placed in the apparatus body 2702. Further, the signal generating device 2704 may be configured for generating at least one signal with at least one signal characteristic.

Further, the at least one field generating element 2706 may be placed in the apparatus body 2702. Further, the at least one field generating element 2706 may be coupled with the signal generating device 2704. Further, the at least one field generating element 2706 may be configured for generating at least one RF magnetic field with at least one RF magnetic field characteristic based on the generating of the at least one signal. Further, the at least one field generating element 2706 may be comprised in the neck strap portion 2710. Further, the at least one RF magnetic field with the at least one RF magnetic field characteristic may be applied to the at least one vagal nerve based on the placing of the apparatus body 2702 and the generating of the at least one RF magnetic field with the at least one RF magnetic field characteristic. Further, the application of the at least one RF magnetic field with the at least one RF magnetic field characteristic may be associated with at least one time period and at least one course for suppressing a chronic inflammatory response in the body of the user.

Further, the at least one powering device 2708 placed in the apparatus body 2702. Further, the at least one powering device 2708 may be electrically coupled with the signal generating device 2704. Further, the at least one powering device 2708 may be configured for powering the signal generating device 2704. Further, the generating of the at least one signal with the at least one signal characteristic may be based on the powering. Further, the signal generating device 2704 and the at least one powering device 2708 may be comprised in the pendant portion 2712.

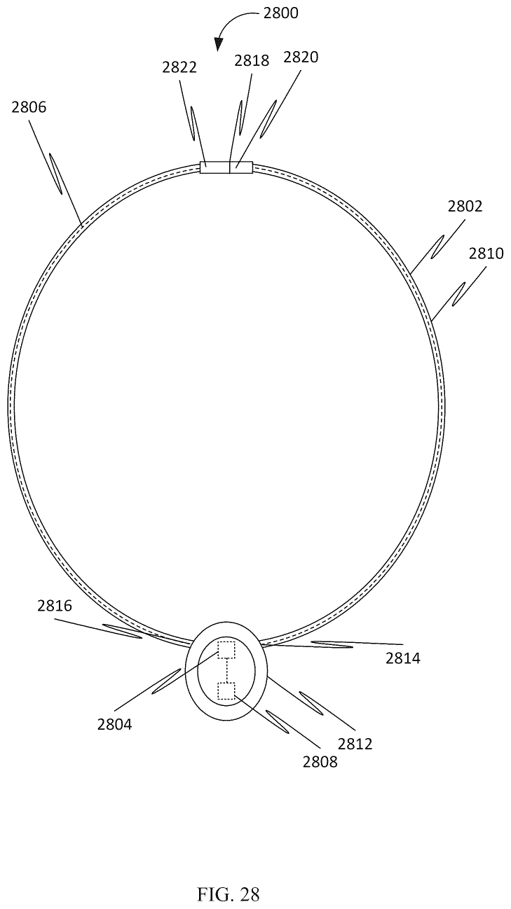

FIG. 28 is a front view of an apparatus 2800 for facilitating stimulating of vagal nerves of the user, in accordance with some embodiments. Accordingly, the apparatus 2800 may include an apparatus body 2802, a signal generating device 2804, at least one field generating element 2806, and at least one powering device 2808.

Further, the apparatus body 2802 may be configured to be placed on at least one part of a body of the user. Further, the at least one part of the body may be associated with at least one vagal nerve. Further, the apparatus body 2802 may include a neck strap portion 2810 and a pendant portion 2812 attached to the neck strap portion 2810. Further, the at least one part of the body may include a neck of the user. Further, the neck strap portion 2810 may be placed around the neck of the user. Further, the neck strap portion 2810 extends between a first end 2814 and a second end 2816. Further, the neck strap portion 2810 may include a connecting element 2818. Further, the connecting element 2818 may be configured for transitioning a loop formed by the neck strap portion 2810 between a continuous loop and a discontinuous loop. Further, the connecting element 2818 may include a first connecting portion 2820 and a second connecting portion 2822. Further, the first connecting portion 2820 may be comprised in the first end of the neck strap portion 2810 and the second connecting portion 2822 may be comprised in the second end 2816 of the neck strap portion 2810. Further, the first connecting portion 2820 may be disconnectably connectable to the second connecting portion 2822 for the transitioning of the neck strap portion 2810 between the continuous loop and the discontinuous loop.

Further, the signal generating device 2804 may be placed in the apparatus body 2802. Further, the signal generating device 2804 may be configured for generating at least one signal with at least one signal characteristic.

Further, the at least one field generating element 2806 may be placed in the apparatus body 2802. Further, the at least one field generating element 2806 may be coupled with the signal generating device 2804. Further, the at least one field generating element 2806 may be configured for generating at least one RF magnetic field with at least one RF magnetic field characteristic based on the generating of the at least one signal. Further, the at least one field generating element 2806 may be comprised in the neck strap portion 2810. Further, the at least one RF magnetic field with the at least one RF magnetic field characteristic may be applied to the at least one vagal nerve based on the placing of the apparatus body 2802 and the generating of the at least one RF magnetic field with the at least one RF magnetic field characteristic. Further, the application of the at least one RF magnetic field with the at least one RF magnetic field characteristic may be associated with at least one time period and at least one course for suppressing a chronic inflammatory response in the body of the user.

Further, the at least one powering device 2808 is placed in the apparatus body 2802. Further, the at least one powering device 2808 may be electrically coupled with the signal generating device 2804. Further, the at least one powering device 2808 may be configured for powering the signal generating device 2804. Further, the generating of the at least one signal with the at least one signal characteristic may be based on the powering. Further, the signal generating device 2804 and the at least one powering device 2808 may be comprised in the pendant portion 2812.

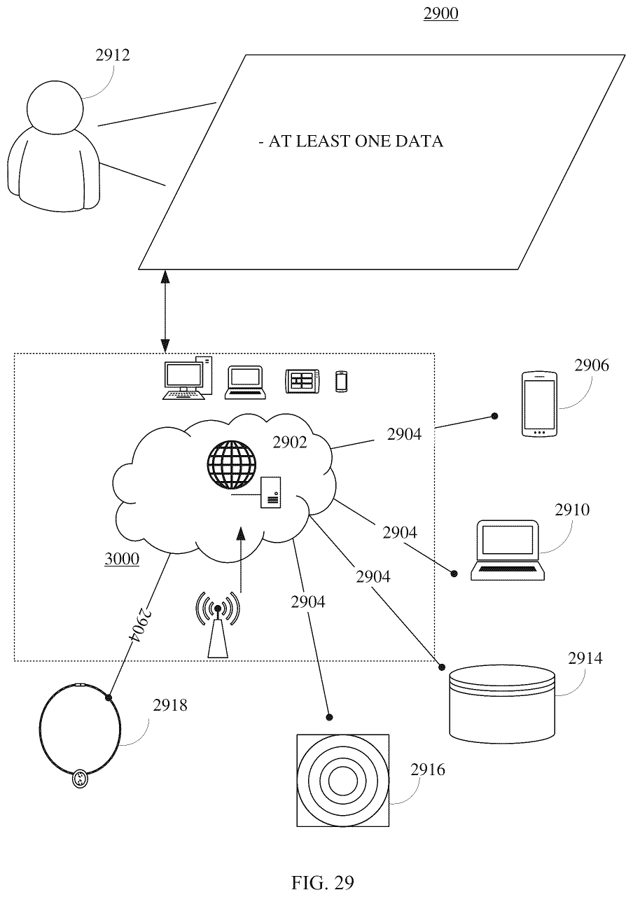

FIG. 29 is an illustration of an online platform 2900 consistent with various embodiments of the present disclosure. By way of non-limiting example, the online platform 2900 to facilitate stimulating of vagal nerves of a user may be hosted on a centralized server 2902, such as, for example, a cloud computing service. The centralized server 2902 may communicate with other network entities, such as, for example, a mobile device 2906 (such as a smartphone, a laptop, a tablet computer, etc.), other electronic devices 2910 (such as desktop computers, server computers, etc.), databases 2914, sensors 2916, and an apparatus 2918 (such as the apparatus 100, apparatus 2700, apparatus 2800, etc.) over a communication network 2904, such as, but not limited to, the Internet. Further, users of the online platform 2900 may include relevant parties such as, but not limited to, end-users, administrators, service providers, and service consumers and so on. Accordingly, in some instances, electronic devices operated by the one or more relevant parties may be in communication with the platform.

A user 2912, such as the one or more relevant parties, may access online platform 2900 through a web based software application or browser. The web based software application may be embodied as, for example, but not be limited to, a website, a web application, a desktop application, and a mobile application compatible with a computing device 3000.

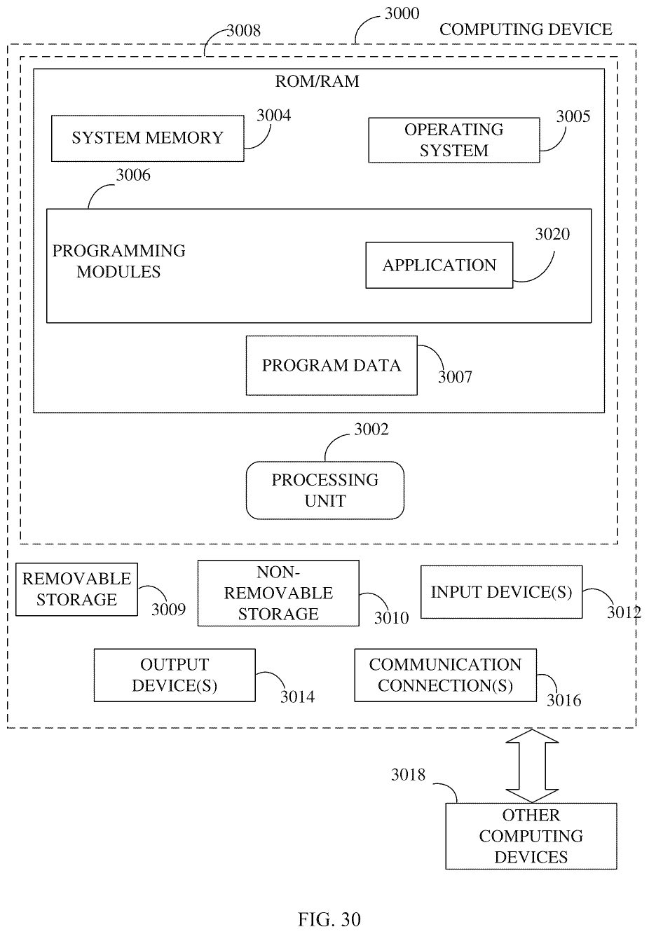

With reference to FIG. 30, a system consistent with an embodiment of the disclosure may include a computing device or cloud service, such as computing device 3000. In a basic configuration, computing device 3000 may include at least one processing unit 3002 and a system memory 3004. Depending on the configuration and type of computing device, system memory 3004 may comprise, but is not limited to, volatile (e.g. random-access memory (RAM)), non-volatile (e.g. read-only memory (ROM)), flash memory, or any combination. System memory 3004 may include operating system 3005, one or more programming modules 3006, and may include a program data 3007. Operating system 3005, for example, may be suitable for controlling computing device 3000's operation. In one embodiment, programming modules 3006 may include image-processing module, machine learning module. Furthermore, embodiments of the disclosure may be practiced in conjunction with a graphics library, other operating systems, or any other application program and is not limited to any particular application or system. This basic configuration is illustrated in FIG. 30 by those components within a dashed line 3008.

Computing device 3000 may have additional features or functionality. For example, computing device 3000 may also include additional data storage devices (removable and/or non-removable) such as, for example, disks, optical disks, or tape. Such additional storage is illustrated in FIG. 30 by a removable storage 3009 and a non-removable storage 3010. Computer storage media may include volatile and non-volatile, removable and non-removable media implemented in any method or technology for storage of information, such as computer-readable instructions, data structures, program modules, or other data. System memory 3004, removable storage 3009, and non-removable storage 3010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 3000. Any such computer storage media may be part of device 3000. Computing device 3000 may also have input device(s) 3012 such as a keyboard, a mouse, a pen, a sound input device, a touch input device, a location sensor, a camera, a biometric sensor, etc. Output device(s) 3014 such as a display, speakers, a printer, etc. may also be included. The aforementioned devices are examples and others may be used.

Computing device 3000 may also contain a communication connection 3016 that may allow device 3000 to communicate with other computing devices 3018, such as over a network in a distributed computing environment, for example, an intranet or the Internet. Communication connection 3016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 3004, including operating system 3005. While executing on processing unit 3002, programming modules 3006 (e.g., application 3020) may perform processes including, for example, one or more stages of methods, algorithms, systems, applications, servers, databases as described above. The aforementioned process is an example, and processing unit 3002 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

According to some embodiments, a method by which a radio frequency (RF) magnetic field can be applied to a region of a neck of a user below an ear of the user so as to ensure exposure of vagal nerves of the user to the RF magnetic field, resulting in a suppression of a chronic inflammatory response in a body of the user is disclosed.

Further, in some embodiments, a therapy of exposing the vagal nerves to the RF magnetic field is applied for preferably 12-24 hours per day, in order to suppress the chronic inflammatory response.

Further, in some embodiments, a device (apparatus) generating the RF magnetic field is utilized for a period of 7-14 days (preferably 8 days) in order to effectively treat the chronic inflammatory response evident at one or more specific locations in the body.

Further, in some embodiments, a target region of the body is the 1-10 cm segment of the vagal nerves along the neck parallel to a trachea of the user.

Further, in some embodiments, a frequency of the RF magnetic field is 5-50 MHz, preferably 27.1 MHz.

Further, in some embodiments, an intensity of the RF magnetic field produces a flux density of 0.1-100 microTesla, preferably about 0.5 microTesla at the site of the vagal nerve.

Further, in some embodiments, the RF magnetic field is pulsed at a pulse rate of 100 Hz to 10,000 Hz, preferably at 1000 Hz.

Further, in some embodiments, one or more pulses of the RF magnetic field have a duty cycle of 10%-50%, preferably 10%.

Further, in some embodiments, an application of the RF magnetic field is directed toward a treatment of inflammatory responses occurring in musculo-skeletal tissues, heart, pancreas, liver, kidney, lung, brain, gastro-intestinal tract, or reproductive system of the user.

FIG. 31 is a graph 3100 illustrating a time course of improvement in average discomfort associated behavior (DAB) level for the study for reversing chronic inflammation in a canine model utilizing pulsed short-wave therapy, in accordance with some embodiments.

Although the present disclosure has been explained in relation to its preferred embodiment, it is to be understood that many other possible modifications and variations can be made without departing from the spirit and scope of the disclosure.

The following is claimed:

1. A method by which a radio frequency (RF) magnetic field can be applied to a region of a neck of a user below an ear of the user so as to ensure exposure of vagal nerves of the user to the RF magnetic field, resulting in a suppression of a chronic inflammatory response in a body of the user.

2. The method of claim 1, wherein a therapy of exposing the vagal nerves to the RF magnetic field is applied for preferably 12-24 hours per day, in order to suppress the chronic inflammatory response.

3. The method of claim 1, wherein a device generating the RF magnetic field is utilized for a period of 7-14 days (preferably 8 days) in order to effectively treat the chronic inflammatory response evident at one or more specific locations in the body.

4. The method of claim 1, wherein a target region of the body is the 1-10 cm segment of the vagal nerves along the neck parallel to a trachea of the user.

5. The method of claim 1, wherein a frequency of the RF magnetic field is 5-50 MHz, preferably 27.1 MHz.

6. The method of claim 1, wherein a magnetic flux density of the RF magnetic field is 0.1-100 microTesla, preferably about 0.5 microTesla at the site of the vagal nerve.

7. The method of claim 1, wherein the RF magnetic field is pulsed at a pulse rate of 100 Hz to 10,000 Hz, preferably at 1000 Hz.

8. The method of claim 1, wherein one or more pulses of the RF magnetic field have a duty cycle of 10%-50%, preferably 10%.

9. The method of claim 1, wherein an application of the RF magnetic field is directed toward a treatment of inflammatory responses occurring in musculo-skeletal tissues, heart, pancreas, liver, kidney, lung, brain, gastro-intestinal tract, or reproductive system of the user.

10. An apparatus for facilitating stimulation of vagal nerves of a user, the apparatus comprising:
an apparatus body configured to be placed on at least one part of a body of the user, wherein the at least one part of the body is associated with at least one vagal nerve;
a signal generating device placed in the apparatus body, wherein the signal generating device is configured for generating at least one signal with at least one signal characteristic;
at least one field generating element placed in the apparatus body, wherein the at least one field generating element is coupled with the signal generating device, wherein the at least one field generating element is configured for generating at least one RF magnetic field with at least one RF magnetic field characteristic based on the generating of the at least one signal, wherein the at least one RF magnetic field with the at least one RF magnetic field characteristic is applied to the at least one vagal nerve based on the placing of the apparatus body and the generating of the at least one RF magnetic field with the at least one RF magnetic field characteristic, wherein the application of the at least one RF magnetic field with the at least one RF magnetic field characteristic is associated with at least one time period and at least one course for suppressing a chronic inflammatory response in the body of the user, wherein the at least one time period may be an extended time period; and
at least one powering device placed in the apparatus body, wherein the at least one powering device is electrically coupled with the signal generating device, wherein the at least one powering device is configured for powering the signal generating device, wherein the generating of the at least one signal with the at least one signal characteristic is based on the powering.

11. The apparatus of claim 10, wherein the apparatus body comprises a neck strap portion and a pendant portion attached to the neck strap portion, wherein the at least one part of the body may include a neck of the user, wherein the neck strap portion is placed around the neck of the user, wherein the at least one field generating element is comprised in the neck strap portion, wherein the signal generating device and the at least one powering device is comprised in the pendant portion.

12. The apparatus of claim 11, wherein the neck strap portion extends between a first end and a second end, wherein the neck strap portion comprises a connecting element, wherein the connecting element is configured for transitioning a loop formed by the neck strap portion between a continuous loop and a discontinuous loop, wherein the connecting element comprises a first connecting portion and a second connecting portion, wherein the first connecting portion is comprised in the first end of the neck strap portion and the second connecting portion is comprised in the second end of the neck strap portion, wherein the first connecting portion is disconnectably connectable to the second connecting portion for the transitioning of the neck strap portion between the continuous loop and the discontinuous loop.

13. The apparatus of claim 12, wherein the at least one field generating element extends between a first end and a second end, wherein the first end of the at least one field generating element is comprised in the first end of the neck strap portion and the second end of the at least one field generating element is comprised in the second end of the neck strap portion, wherein the transitioning of the loop formed by the neck strap portion between the continuous loop and the discontinuous loop transitions the loop formed by the at least one field generating element between the continuous loop and the discontinuous loop, wherein the generating of the at least one RF magnetic field with the at least one RF magnetic field characteristic is further based on the transitioning of the loop formed by the at least one field generating element to the continuous loop.

14. The apparatus of claim 12, wherein the at least one field generating element extends between a first end and a second end, wherein the first end of the at least one field generating element is comprised in the first end of the neck strap portion and the second end of the at least one field generating element is comprised in the second end of the neck strap portion, wherein the neck strap portion comprises a loop tightening mechanism coupled to the first end of the neck strap portion and the second end of the neck strap portion, wherein the loop tightening mechanism is configured modifying a length of the loop of the neck portion and the at least one field generating element based on at least one action receivable by a tightening element of the loop tightening mechanism, wherein the modifying of the length comprises connectedly overlapping at least one length of at least one of a first portion of the neck strap portion and the at least one field generating element over a second portion of the neck strap portion and the at least one field generating element.

15. The apparatus of claim 11, wherein the neck strap portion is comprised of at least one elastic material, wherein the at least one field generating element comprises a filament, wherein the filament is arranged in at least one configuration, wherein the neck strap portion and the at least one field generating element is configured to be transitioned from an original state to at least extended state by applying at least one external force on the neck strap portion based on the at least one elastic material and the at least one configuration of the filament, wherein the neck strap portion and the at least one field generating element are transitionable from the at least one extended state to the original state by removing the at least one external force from the neck strap portion based on the at least one elastic material and the at least one configuration of the filament.

16. The apparatus of claim 10, wherein the at least one field generating element comprised of at least one electrically conducting material, wherein the at least one field generating element is characterized by a length and a cross sectional area, wherein the generating of the at least one RF magnetic field with the at least one RF magnetic field characteristic is further based on the at least one electrically conducting material, the length, and the cross sectional area of the at least one field generating element.

17. The apparatus of claim 16, wherein the at least one field generating element extends between a first end and a second end, wherein the first end of the at least one field generating element and the second end of the at least one field generating element are coupled to the signal generating device for forming at least one loop, wherein the generating of the at least one RF magnetic field with the at least one RF magnetic field characteristic is further based on the forming of the at least one loop.

18. The apparatus of claim 17, wherein the at least one loop comprises a plurality of loops, wherein the plurality of loops are interconnected.

19. The apparatus of claim 10, wherein the apparatus body is further configured to be attached to a surface of the at least one part of the body of the user using at least one attaching element, wherein the disposing of the apparatus body on the at least one part of the body is further based on the attaching of the apparatus body to the at least one part of the body.

20. The apparatus of claim 10 further comprising a processing device comprised in the apparatus body, wherein the processing device is configured for:
analyzing at least one data;
determining at least one value for at least one parameter associated with the generating of the at least one signal with the at least one signal characteristic based on the analyzing of the at least one data; and
generating at least one command for the signal generating device based on the determining, wherein the signal generating device is operatively coupled with the processing device, wherein the generating of the at least one signal with the at least one characteristic is further based on the at least one command.

21. The apparatus of claim 20 further comprising at least one communication interface comprised in the apparatus body, wherein the at least one communication interface is communicatively coupled with the processing device, wherein the at least one communication device is configured for receiving the at least one data from at least one device.

22. The apparatus of claim 20 further comprising at least one sensor comprised in the apparatus body, wherein the at least one sensor is communicatively coupled with the processing device, wherein the at least one sensor is configured for imaging the at least one field generating element and the at least one vagal nerve, wherein the processing device is further configured for:
generating an imaging data based on the imaging;
analyzing the imaging data; and
determining a proximity between the at least one field generating element and the at least one vagal nerve based on the analyzing of the imaging data, wherein the determining of the at least one value for the at least one parameter is further based on the proximity.

23. The apparatus of claim 20 further comprising at least one biological sensor comprised in the apparatus body, wherein the at least one biological sensor is communicatively coupled with the processing device, wherein the at least one biological sensor is configured for detecting a characteristic of the at least one part of the body, wherein the processing device is further configured for:
generating a biological data based on the detecting of the characteristic; and
analyzing the biological data, wherein the determining of the at least one value for the at least one parameter is further based on the analyzing of the biological data.

24. The apparatus of claim 10 further comprising:
at least one position sensor comprised in the apparatus body, wherein the at least one position sensor is communicatively coupled with the processing device, wherein the at least one position sensor is configured for detecting a position of the at least one field generating element in relation to the at least one vagal nerve;
a processing device comprised in the apparatus body, wherein the processing device is communicatively coupled with the at least one position sensor, wherein the processing device is configured for:
generating a position data based on the detecting of the position;
analyzing the position data based on at least one criterion;
determining an alignment of the at field generating element in relation to the at least one vagal nerve based on the analyzing of the position data; and
generating an output signal corresponding to the alignment based on the determining; and
at least one output device communicatively coupled with the processing device, wherein the at least one output device is configured for generating at least one output based on the output signal.

25. The apparatus of claim 10, wherein the apparatus body extends between a first end and a second end, wherein the apparatus body is curvedly shaped with a curvature for defining an interior space, wherein the apparatus body comprises a first body portion comprising the first end of the apparatus body and a second body portion comprising the second end of the apparatus body, wherein the first body portion opposes the second body portion, wherein the at least one part of the body comprises a head of the user, wherein at least one of the first body portion and the second body portion is transitionable from an original state to at least one extended state for transitioning the curvature from an original curvature to at least one extended curvature based on an application of at least one external force, wherein the apparatus body receives at least a portion of the head of the user in the interior space based on the transitioning of the curvature to the at least one extended curvature, wherein the first body portion and the second body portion transition to at least one retracted state from the at least one extended state for transitioning the curvature from the at least one extended curvature to at least one retracted curvature based on a removal of the at least one external force, wherein the first body portion and the second body portion presses against at least the portion of the head in the at least one retracted state for securing the apparatus body to the head, wherein the disposing of the apparatus body to the at least one part of the body is further based on the securing.

26. The apparatus of claim 10 further comprising at least one output device placed in the apparatus body, wherein the at least one output device is operatively coupled with the signal generating device, wherein the at least one output device is configured for outputting at least one stimulus perceptible to the user based on the generating of the at least one signal with the at least one signal characteristic.

27. The apparatus of claim 10 further comprising a wearable apparel configured to be worn on the at least one part of the body, wherein the wearable apparel comprises at least one holder, wherein the at least one holder is configured for securing the apparatus body in at least one location on the wearable apparel, wherein the disposing of the apparatus body on the at least one part of the body is further based on the securing of the apparatus body in the at least one location and the wearing of the wearable apparel.

28. An apparatus for facilitating stimulating of vagal nerves of a user, the apparatus comprising:
an apparatus body configured to be placed on at least one part of a body of the user, wherein the at least one part of the body is associated with at least one vagal nerve, wherein the apparatus body comprises a neck strap portion and a pendant portion attached to the neck strap portion, wherein the at least one part of the body may include a neck of the user, wherein the neck strap portion is placed around the neck of the user;
a signal generating device placed in the apparatus body, wherein the signal generating device is configured for generating at least one signal with at least one signal characteristic;
at least one field generating element placed in the apparatus body, wherein the at least one field generating element is coupled with the signal generating device, wherein the at least one field generating element is configured for generating at least one RF magnetic field with at least one RF magnetic field characteristic based on the generating of the at least one signal, wherein the at least one field generating element is comprised in the neck strap portion, wherein the at least one RF magnetic field with the at least one RF magnetic field characteristic is applied to the at least one vagal nerve based on the placing of the apparatus body and the generating of the at least one RF magnetic field with the at least one RF magnetic field characteristic, wherein the application of the at least one RF magnetic field with the at least one RF magnetic field characteristic is associated with at least one time period and at least one course for suppressing a chronic inflammatory response in the body of the user, wherein the at least one time period may be an extended time period; and
at least one powering device placed in the apparatus body, wherein the at least one powering device is electrically coupled with the signal generating device, wherein the at least one powering device is configured for powering the signal generating device, wherein the generating of the at least one signal with the at least one signal characteristic is based on the powering, wherein the signal generating device and the at least one powering device is comprised in the pendant portion.

29. An apparatus for facilitating stimulating of vagal nerves of a user, the apparatus comprising:
an apparatus body configured to be placed on at least one part of a body of the user, wherein the at least one part of the body is associated with at least one vagal nerve, wherein the apparatus body comprises a neck strap portion and a pendant portion attached to the neck strap portion, wherein the at least one part of the body may include a neck of the user, wherein the neck strap portion is placed around the neck of the user, wherein the neck strap portion extends between a first end and a second end, wherein the neck strap portion comprises a connecting element, wherein the connecting element is configured for transitioning a loop formed by the neck strap portion between a continuous loop and a discontinuous loop, wherein the connecting element comprises a first connecting portion and a second connecting portion, wherein the first connecting portion is comprised in the first end of the neck strap portion and the second connecting portion is comprised in the second end of the neck strap portion, wherein the first connecting portion is disconnectably connectable to the second connecting portion for the transitioning of the neck strap portion between the continuous loop and the discontinuous loop;
a signal generating device placed in the apparatus body, wherein the signal generating device is configured for generating at least one signal with at least one signal characteristic;
at least one field generating element placed in the apparatus body, wherein the at least one field generating element is coupled with the signal generating device, wherein the at least one field generating element is configured for generating at least one RF magnetic field with at least one RF magnetic field characteristic based on the generating of the at least one signal, wherein the at least one field generating element is comprised in the neck strap portion, wherein the at least one RF magnetic field with the at least one RF magnetic field characteristic is applied to the at least one vagal nerve based on the placing of the apparatus body and the generating of the at least one RF magnetic field with the at least one RF magnetic field characteristic, wherein the application of the at least one RF magnetic field with the at least one RF magnetic field characteristic is associated with at least one time period and at least one course for suppressing a chronic inflammatory response in the body of the user, wherein the at least one time period may be an extended time period; and
at least one powering device placed in the apparatus body, wherein the at least one powering device is electrically coupled with the signal generating device, wherein the at least one powering device is configured for powering the signal generating device, wherein the generating of the at least one signal with the at least one signal characteristic is based on the powering, wherein the signal generating device and the at least one powering device is comprised in the pendant portion.
